(19) United States
(12) Patent Application Publication
(10) Pub. No.: US 2021/0330696 A1
(43) Pub. Date: Oct. 28, 2021

(54) A HIGH CAPACITY PLATFORM FOR IMMUNOGENIC CANCER CELL DEATH

(71) Applicants: Rita Elena Serda, Pearland, TX (US); Achraf Noureddone, Albuquerque, NM (US)

(72) Inventors: Rita Elena Serda, Pearland, TX (US); Achraf Noureddone, Albuquerque, NM (US)

(21) Appl. No.: 16/978,418

(22) PCT Filed: Mar. 5, 2019
(86) PCT No.: PCT/US2019/020824

§ 371 Date: Sep. 4, 2020

Related U.S. Application Data
(60) Provisional application No. 62/639,198, filed on Mar. 6, 2018.

(51) Int. Cl. A61K 31/739; A61K 31/704; C07K 16/28; A61K 38/20

(52) U.S. Cl. CPC A61K 31/739; A61K 31/704; B82Y 5/00; A61K 38/208; C07K 16/2818

(57) ABSTRACT

Compositions comprising, and methods of using, mesoporous hybrid siliceous nanoparticles comprising a TLR ligand or other immune stimulant(s), and an agent to induce immunogenic cell death.

### CROSS-REFERENCE TO RELATED APPLICATIONS

This application claims the benefit of the filing date of U.S. Provisional Application No. 62/639,198, filed on Mar. 6, 2018, the disclosure of which is incorporated by reference herein.

### BACKGROUND

According to the American Cancer Society, one in eight American women will develop breast cancer in their lifetime, with an estimated 316,120 women in the United States diagnosed with breast cancer and 40,610 women dying from this disease in 2017 alone. In advanced cancer cases, the use of chemotherapy may he palliative, aimed at alleviating symptoms rather than being curative. It is the goal of the National Cancer Institute and researchers to eliminate cancer cells throughout the body in all patients.

Macrophages, which home to sites of inflammation, have been shown to carry nanoparticles (NPs) across the blood-brain-barrier (BBB) and into glioma. Valable et al. (2008) used non-invasive imaging to track the migration of systemically injected monocytes across the BBB into glioma. Pang et al. (2016) demonstrated that RAW macrophages carrying DOX-loaded NPs did not damage the carrier cells when DOX was less than 25 μg/ml. They further showed that tumor targeting of NPs in nude mice was increased when NPs were preloaded in macrophages as compared to direct injection. Li et al, (2017) demonstrated that Nano DOX-loaded tumor-associated macrophages (TAM) were viable and able to infiltrate glioma spheroids, releasing their drug cargo and causing the release of damage-associated molecular patterns (DAMPS) from cancer cells that surpassed that released in the presence of free DOX. DAMP release further increased tumor infiltration by both free and macrophage-loaded NPs. Alizadeh et al. (2010) demonstrated that TAMs migrate between distant tumors in mice.

### SUMMARY

NPs are proviced that target macrophages and other antigen presenting cells (APC), activating the APC with Toll-like receptor ligands, cytokines or other immunostimulatory molecules, and carrying ICD-inducing chemotherapeutics that when released in the tumor microenvironment lead to immunogenic cell death of cancer cells. In one embodiment, ICD-inducing chemotherapy-loaded mesoporous silica cores are encapsulated with adjuvant-modified lipid bilayers for targeting antigen presenting and cancer cells. ICD chemotherapeutics sensitize tumors to T cell mediated immune responses by triggering immunogenic cell death, e.g., that is dependent on TLR-4 and CD8⁺ T cells. In particular, a NP-based platform is provided that buildsnon the ability of select chemotherapy agents to induce immunogenic cell death (ICD; i.e. regulated cell death, capable of activating an adaptive immune response against cell-associated antigens). Microbial products, known as pathogen-associated molecular patterns (PAMPs), stimulate APC maturation through TLR or other pattern recognition receptors (PRRs), promoting expression of co-stimulatory molecules and cytokines. In one embodiment, immunogenic mesoporous hybrid siliceous nanoparticles (MHySN; e,g., mesoporous bridged silsesquioxanes, including MHySNs with pH dependent release of cargos including but not limited to chemotherapeutic agents) are provided for logic-embedded, sequential presentation of an immunostimulatory molecule, e.g., a TLR4 ligand (for activation of immune cells and targeting of receptors on the surface of immune cells), followed by an ICD-inducing chemotherapeutic agent, with or without a second immunostimulatory molecule such as a TLR ligand (e.g., a TLR9 ligand), MHySN are formed by liposome fusion on high surface area (>500 m²/g) MHySN cores. The use of these nanoparticles allows for: 1) immunogenic presentation of antigen arising from chemotherapy-induced apoptosis; 2) stimulation of TLR signaling pathways in phagocytic antigen presenting cells (APC), optionally two or more TLR signaling pathways; 3) immune cell targeting, and/or 4) optionally exhibition of a silsesquioxane core with outstanding functional fertility and controllable drug release properties. In one embodiment, chemotherapy-induced immunogenic cell death is augmented using nanomedicine to co-deliver the ICD-inducing chemotherapeutic agent doxorubicin (DOX) hydrochloride and bacterial Toll-like receptor (TLR) ligands combined with immune checkpoint blockade.

In one embodiment, a mesoporous hybrid siliceous nanoparticle comprising an immune stimulant such as a TLR4 ligand and an agent to induce immunogenic cell death is provided. In one embodiment, the nanoparticle further comprises a different immune stimulant, e.g., a TLR Ilgand. In one embodiment, the different TLR ligand is a TLR9 ligand. In one embodiment, the TLR9 ligand comprises CpG oligonucleotides. In one embodiment, the CpG oligonucleotide comprises a phosphodiester (PO) central CpG-containing palindromic motif and a phosphorothioate (PS)-modified 3′ poly-G string. In one embodiment, the CpG oligonucleotide comprises a full PS backbone with one or more CpG dinucleotides. In one embodiment, the CpG oligonucleotide comprises a complete PS backbone and a CpG-containing palindromic motif. In one embodiment, the TLR9 ligand comprises SD-101, AS15, GNKG168, PF-3512676, ISS 1018, IMO-2055, CpG-28, EMD120108, or BCG. In one embodiment, the TLR4 ligand comprises monophosphoryl lipid (MPL)-A, aminoalkyl glucosaminide phosphate (AGP), Glucopyranosyl Lipid-A, LPS, beta-defensin 2, fibronectin EDA, HMGB1, AS15, snapin, tenascin C, ER111232, ER111233, ER112040, ER111230, ER112231, ER112093, ER112049, ER112047, ER112066, ER113651, ER119327, ER803022, ER803732, or ER803789. In one embodiment, the agent that induces ICD comprises an anthracycline, R2016 (3-(4-chlorophenylamino)-6-hydroxy-9-methyl-9H-carbazole-1,4-dione), an anthracenedione, a platinum compound, an alkylating agent, proteasomal inhibitor, or immunogenic cell-killing RNA, e.g., ICR2 and ICRif. In one embodiment, the agent that induces ICD comprises doxorubicin, epirubicin, idarubicin, mitoxantrone, oxaliplatin, cyclophosphamide, or bortezomib. In one embodiment, the agent that induces ICD is linked to the silaceous core. In one embodiment, the linker is pH sensitive, light sensitive, redox sensitive or comprises a hydrazine or benzene-bridged silsesquioxane. In one embodiment, the nanoperticle of has a diameter of about 50 nm to about 150 nm. In one embodiment, the nanoparticle further comprises a lipid layer, e.g., a lipid bi-layer, thereby forming a “protocell”. In one embodiment, the core is pH sensitive, light sensitive, redox sensitive or comprises a benzene-bridged silsesquioxane. In one embodiment, the nanopailicle has pores of about 5 to 20 nm in diameter or about 8 to 15 nm in diameter. Further provided is a pharmaceutical composition comprising a population of the nanoparticles, and optionally further comprising an anti-PD1 agent, e.g., an anti-PD1 antibody.

In one embodiment, a method to stimulate antitumor immunity, activate dendritic cells (DCs), or stimulate antigen processing presentation in a mammal is provided. In one embodiment, a mammal is administered an effective amount of a plurality of the nanoparticles and optionally an immune checkpoint inhibitor such as an anti-PD-1 or PD-L1 agent, e.g., dinaciclib, cemiplimab, nivolumab, pembrolizumab, pidilizumab, BMS-936559, MPDL3280A, MED14736, MSB0010718C, avelumab, durvalumab, or atezolizumab. In one embodiment, the nanoparticle comprises the anti-PD1 agent. In one embodiment, the lipid layer comprises the checkpoint

Also provided is a method of using ICD inducing chemotherapeutics to elicit immunogenic apoptotic bodies from cancer cells to use as vaccines.

In one embodiment, MHySN vaccines are administered to a mammal, such as a human, via intravenous, intraperitoneal or intratumoral routes. The vaccine provides for logic-embedded drug presentation, e.g., sequential presentation of an immune stimulant such as a TLR-4 ligand (for activation of immune cells and targeting of receptors on the surface of immune cells), followed by an ICD (immunogenic cell death)-inducing chemotherapeutic agent, with or without another immune stimulant, e.g., a TLR ligand such as a TLR-9 ligand. MHySN are formed by liposome fusion on high surface area (>500 m²/g) MHySN cores.

In one embodiment, immunogenic mesoporous hybrid siliceous nanoparticles (MHySN) deliver an immunogenic cell death inducing molecule and an adjuvant that is a TLR ligand, and optionally a second (different) TLR ligand and/or optionally mesoporous bridged silsesquioxanes, which particles optionally exhibit pH dependent release of cargo.

### BRIEF DESCRIPTION OF FIGURES

FIG. 1. Engineering immunogenic cell death (ICD)-inducing nanoparticles for cancer therapy. Mesoporous cores, composed of either silica or silsesquioxane, are loaded with drug and then coated with a lipid coat by fusion of liposomes onto the core surface. When labeled with a fluorescent core, particles can be visualized inside the target cell using fluorescent microscopy (red are nanoparticles and blue is the cacner cell nuclei).

FIG. 2. Hallmarks of immunogenic cell death (ICD) via drug-laden nanoparticles. The schematic shows fusion of liposomes (green circles) onto the surface of a mesoporous silica core. When the nanoparticles are loaded with ICD-inducing chemotherapeutics, cancer cells that have internalized the nanoparticles perform 3 hallmarks representative of ICD: 1) translocation of calreticulin to the cell surface (eat me); 2) release of ATP (find me); and 3) release of HMGB1 (TLR-4 ligand).

FIG. 3. Nanoparticle Synthesis: Mesoporous Hybrid Siliceous Nanoparticle (MHySN). Materials and their molar ratios needed to create pure silica or organosilica cores are presented. Transmission electron micrographs of each core type are shown.

FIG. 4. Cargo: ICD-inducers and adjuvant. Drug cargo that we have tested in our mesoporous hybrid siliceous nanoparticles include cisplatin (2 hallmarks); and oxalipltin or doxorubicin (each stimulate 31CD hallmarks). Adjuvants included in the nanoparticles includes MPL or CpG, but other Toll-like receptor ligands, or other PAMP, DAMP, cytokine, or small molecule are candidates for inclusion as adjuvant.

FIG. 5. Liposomal Formulations. Three liposome formulations tested to create mesoporous hybrid siliceous nanoparticles are presented in the table. The resulting nanoparticle size (dynamic light scattering) and charge (zeta potential) are presented graphically. All formulations tested contained DOTAP to create a cationic surface to encourage uptake by cancer cells.

FIG. 6. Hydrodynamic Size of drug-free and drug-loaded LC-MHySN. The impact of drug loading on nanoparticle size is shown for lipid coated silica and benzene organosilane nanoparticles loaded with oxaliplatin or doxorubicin.

FIG. 7. Lipid fusion on silica cores. The drawings show circular liposome fusing onto mesoporous silica cores. The resulting LC-MHySNs were stained using Phosphotungstic acid and imaged by transmission electron microscopy.

FIG. 8. Lipid fusion on MHySN: Zeta Potential. Surface charge of nanoparticles during each synthesis step is shown graphically. Both organosilica and silica (MSN) cores are negative. After fusion with cationic liposomes (LC-organosilica and LC-MSN), the nanoparticle zeta potential shift to neutral values.

FIG. 9. Drug loaded LC-MHySN: 1D-8 cytotoxicity studies. The impact of unloaded or drug-loaded MHySN on cancer cell proliferation is shown. An alamar blue assay was used to measure proliferation after 1D8 ovarian cancer cells were incubated with MHySN for 24 h at 25 μg/ml. MHySN loaded with either oxaliplatin (OXA) or doxorubicin (DOX) decreased cell growth.

FIG. 10. Imaging ID8 ovarian cancer cells after uptake of MHySN. Fluorescent confocal micrographs show ID8 cells (blue nuclei) 24 h after addition of cisplatin or doxorubicin loaded RITC (red fluorescent)-MHySN to the cell culture at 25 μg/ml. Nuclei in cells treated with doxorubicin loaded MHySN are purple based on the presence of DAPI (blue) and doxorubicin (red).

FIG. 11. LC-MHySN: cell internalization kinetics. Time-dependent internalization of fluorescent-labeled (cyanine-3; red) MHySN in IB8 ovarian cancer cells (DAPI; blue nuclei). Cells are located in the perinuclear region of the cell based on localization within endosomes.

FIG. 12. SEM image of T cells (red) attacking a cancer cell (white). 4T1 tumors from PBS control, empty liposomes, MPL-liposomes, IL-12 and NANO-MPL-12 treated mice.

FIGS. 13A-D. Anti-PD-1 checkpoint inhibitor antibody causes tumor growth and morbidity in BALB/c mice with 4T1 tdTomato red luc tumors. A) 4T1 tdTomato red/luc tumor bioluminescence before and post NP and anti-PD-1 treatment (2D and 3D). B) Weight and growth curves for mice with 4T1 luc or 4T1 tdTomato red luc tumors. C) SEM images of tumor tissues in control and anti-PD1 antibody treated mice. D) H&E.

FIGS. 14A-B. Multi-modal imaging of 4T1 tumors. A) Cancer cell location (bioluminescence), metabolic activity (¹⁸F-FDG PET), and vascular perfusion of the tumor (DCE-MRI). B) Tumor vascular architecture (CT and fluorescent intravital imaging) and vessel modeling.

FIGS. 15A-F. pH-dependent DOX release and APC activation by MHySN, A) 3D surface-rendered confocal image of DOX (green)-nucleic acid (red) loaded MHySN near a cell nucleus (blue). B) Upregulation of CD40 in macrophages by MPL MHySN comparing unmodified or COOH silica core and lipids made by extrusion (Ex) or sonication. C) TEM image of mesoporous silsesquioxane nanoparticles. D) DOX release profile of the particles upon pH trigger. E-F) Fluorescent quantitation of the pH-triggered DOX release.

FIGS. 16A-C. Intercellular transport of NPs and DOX between homo and heterotypic cells. A) Drawing of TNT intercellular transfer of NPs and SEM images of MSN transfer between macrophages. B) SEM images of intercellular communication between macrophages and cancer cells. C) DOX (red) transfer between macrophages.

FIG. 17. MR imaging of 4T1 tumor infiltration by myeloid cells. The perfluorocarbon emulsion V-Sense was injected intravascularly and carrier macrophages were imaged by 19F MRI 48 h post control or IL-12 intraturrioral injection.

FIG. 18. Schematic of MHySN fabrication. Mesoporous siliceous cores, co-loaded with DOX and CpG ODN, are surrounded by an MPL containing supported lipid bilayer.

FIG. 19. Macrophages as NP carriers to the tumor. A) SPECT images of a mouse injected i.v. with ¹¹¹In-NPs as a function of time. B) NP accumulation in tumor compared to filtering organs based on direct NP injection vs adoptive transfer of macrophages preloaded with NPs.

FIGS. 20A-B. Impact of NPs on tumor growth and the tumor microenvironment. Influence of weekly NP injections on A) tumor growth, B) Th-1 cytokines, tumor-associated proliferation (Ki-67), DC (33D1), and CD8⁺ T cells.

FIGS. 21A-B. Tumor immunocytes. A) Flow cytometry dot blots showing gating for myeloid and T cell populations. B) Percent immunocytes in control vs IL-12 treated tumors.

FIG. 22. NP modulation of the tumor microenvironment. BALB/c mice with 4T1 breast tumors were treated with control or MPL loaded liposomes. The impact of NPs on CD8⁺ T cells (red), macrophages [F4/80 (green) and CD204 (red)], DC (33D1; red), and INOS (green) in tumors is shown (nuclei blue).

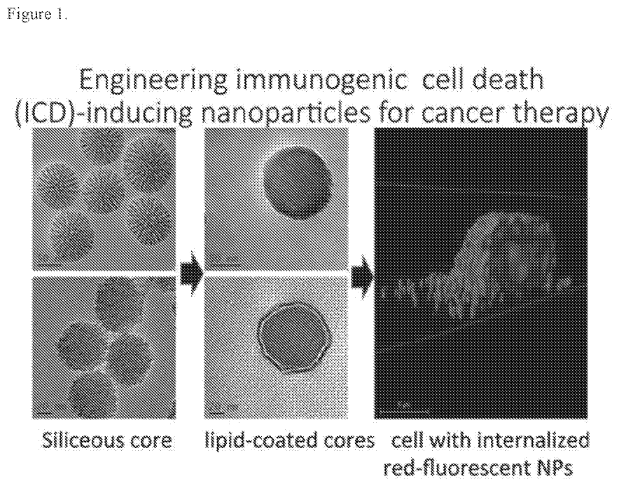

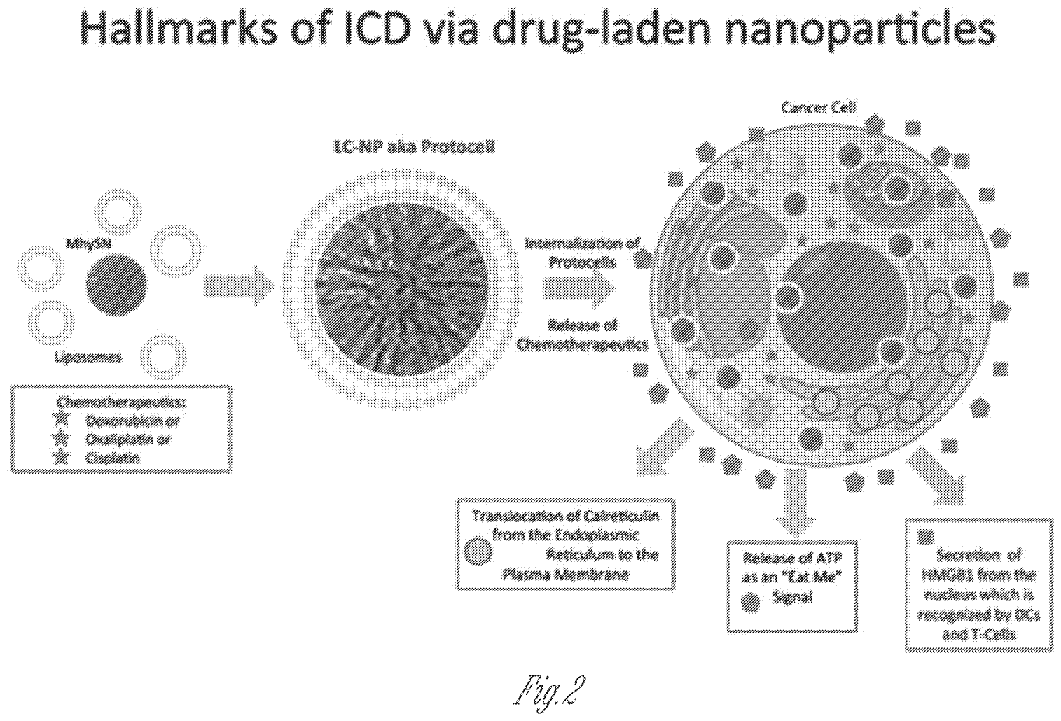

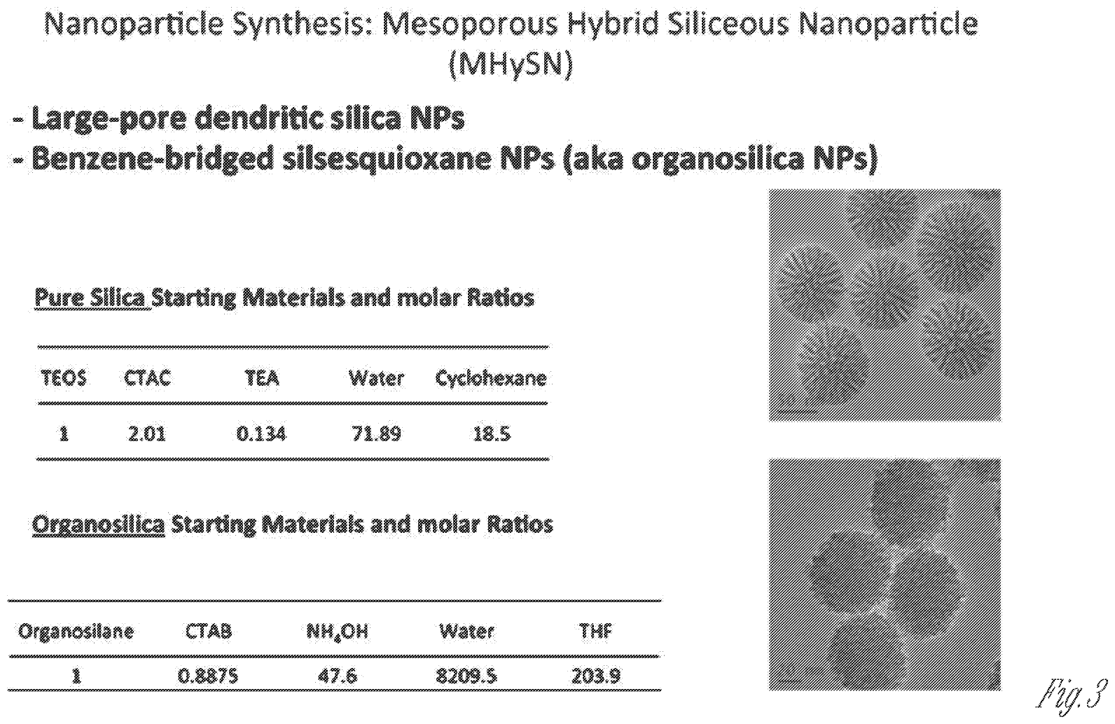

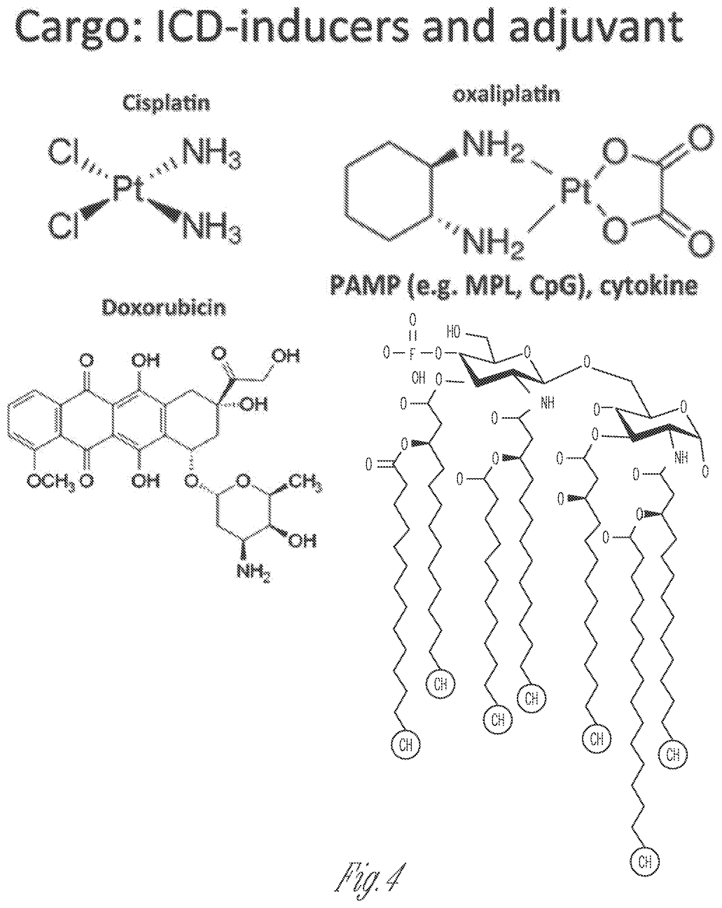

### DETAILED DESCRIPTION

### Definitions

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, a prism or a whisker. A nanoparticle may include particles having two or more of the aforementioned shapes. In one embodiment, a cross-sectional geometry of the particle may be one or more of circular, ellipsoidal, triangular, toroidal, rectangular or polygonal. In one embodiment, a nanoparticle may consist essentially of non-spherical particles, especially prisms. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles. In one embodiment, a plurality of nanoparticles may consist essentially of hexagonal prism nanoparticles.

The term “monosized protocells” is used to describe a population of monosized (monodisperse) protocells comprising a lipid bi-layer fused onto a mMSNPs as otherwise described herein. In some embodiments, monosized protocells are prepared by fusing the lipids in monosized unilamellar liposomes onto the mMSNPs in aqueous buffer (e.g., phosphate buffered solution) or other solution at about room temperature, although slightly higher and lower temperatures may be used. The unilamellar liposomes which are fused onto the mMSNPs are monodisperse with hydrodynamic diameters of, in one example, less than about 100 nm, often about 65-95 nm, most often about 90-95 nm, although unilamellar liposomes which can be used may fall outside this range depending on the size of the mMSNPs to which lipids are to be fused and low PDI values (generally, less than about 0.5, e.g., less than 0.2). The mass ratio of liposomes to mMSNPs used to create monosized protocells which have a single lipid bi-layer completely surrounding the mMSNPs is that amount sufficient to provide a liposome interior surface area which equals or exceeds the exterior surface area of the mMSNPs to which the lipid is to be fused. This often is provided in a mass ratio of liposomes to mMSNPs of at least about 2:1, often up to about 10:1 or more, with a range often used being about 2:1 to about 5:1. The resulting protocells are monosized (monodisperse). Monosized protocells may exhibit extended storage stability in aqueous solution, e.g., providing a SLB on the protocell which has a transition temperature Tₘ which is greater than the storage, experimental or administration/therapeutic conditions under which the protocells are stored and/or used. Often the protocell is at least about 25-30 nm in diameter larger than the diameter of the mMSNPs.

The phrase “effective average particle size” as used herein to describe a multiparticulate (e.g., a porous nanoparticulate) means that all particles therein are of an average diameter or within ±5% of the average diameter. In certain embodiments, nanoparticulates have an effective average particle size (diameter) of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 rim, less than about 200 nm, less than about 150 nm, less than about 100 nm, less than about 75 nm, less than about 50 nm, less than about 35 nm, less than about 25 nm, as measured by light-scattering methods, microscopy, or other appropriate methods. In exemplary aspects, the average diameter of SMSNPs ranges from about 75 nm to about 150 nm, often about 75 to about 130 nm, often about 75 nm to about 100 nm.

The term “patient” or “subject” is used throughout the specification within context to describe an animal, generally a mammal, especially including a domesticated animal or mammal and for example a human, to whom treatment, including prophylactic treatment (prophylaxis), with the compounds or compositions is provided. For treatment of those infections, conditions or disease states which are specific for a specific animal such as a human patient, the term patient refers to that specific animal. In most instances, the patient or subject is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The terms “treat”, “treating”, and “treatment”, are used synonymously to refer to any action providing a benefit to a patient at risk for or afflicted with a disease state or condition, including improvement in the disease state or condition through lessening, inhibition, suppression or elimination of at least one symptom, delay in progression of the disease, prevention, delay in or inhibition of the likelihood of the onset of the disease state and/or condition, etc. In the case of microbial infections, these terms also apply to microbial (e.g., viral or bacterial) infections and may include, in certain particularly favorable embodiments the eradication or elimination (as provided by limits of diagnostics) of the microbe (e,g., a virus or a bacterium) which is the causative agent of the infection.

Treatment, as used herein, encompasses both prophylactic and therapeutic treatment, e.g., of cancer (including inhibiting metastasis or recurrence of a cancer in remission). Compounds can, for example, be administered prophylactically to a mammal in advance of the occurrence of disease to reduce the likelihood of that disease. Prophylactic administration, e.g., a vaccine, is effective to reduce or decrease the likelihood of the subsequent occurrence of disease in the mammal, or decrease the severity of disease (inhibition) that subsequently occurs, especially including metastasis of cancer. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds is effective to decrease the severity of the disease or lengthen the lifespan of the mammal so afflicted, as in the case of cancer, or inhibit or even eliminate the causative agent of the disease.

The term “prophylactic administration” refers to any action in advance of the occurrence of disease to reduce the likelihood of that disease or any action to reduce the likelihood of the subsequent occurrence of disease in the subject. Compositions can, for example, be administered prophylactically to a mammal in advance of the occurrence of disease to enhance an immunogenic effect and/or reduce the likelihood of that disease. Prophylactic administration is effective to reduce or decrease the likelihood of the subsequent occurrence of disease in the mammal, or decrease the severity of disease (inhibition) that subsequently occurs, especially cancer, its metastasis or recurrence.

The term “targeting active species” is used to describe a compound or moiety which is complexed or covalently bonded to the surface of a protocell which binds to a moiety on the surface of a cell to be targeted so that the protocell may selectively bind to the surface of the targeted cell and deposit its contents into the cell. In one embodiment, the targeting active species is a “targeting peptide” including a polypeptide including an antibody or antibody fragment, an aptamer, or a carbohydrate, among other species which bind to a targeted cell. A targeting active species may be peptide of a particular sequence which binds to a receptor or other polypeptide in cancer cells and allows the targeting of protocells to particular cells which express a peptide (be it a receptor or other functional polypeptide) to which the targeting peptide binds. Targeting peptides may be complexed or covalently linked to the lipid bi-layer through use of a crosslinking agent as otherwise described herein.

The terms “fusogenic peptide” and “endosomolytic peptide” are used synonymously to describe a peptide which is optionally crosslinked onto the lipid bi-layer surface of the protocells. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative fusogenic peptides for use in protocells include but are not limited to H5WYG peptide, H₂N-GLFHAIAHFIHGGWHGLIHGWYGGC-COOH (SEQ ID. NO:2) or an 8 merpolyarginine (H₂N-RRRRRRRR-COOH, SEQ ID NO:3), among others known in the art. Additional fusogenic peptides include RALA peptide (NH₂-WEARLARALARALARHLARALARALRAGEA-COOH, SEQ ID NO: 4), KALA peptide (NH₂-WEAKLAKALAKALAKHLAKALAKALKAGEA-COOH), SEQ ID. NO:5), GALA (NH2-WEAALAEALAEALAEHLAEALAEALEALAA-COOH, SEQ ID NO:6) and INF7 (NH2-GLFEAIEGFIENGWEGMIDGWYG-COOH, SEQ ID. NO:7), among others.

Thus, the terms “cell penetration peptide,” “fusogenic peptide” and “endosomolytic peptide” are used to describe a peptide which aids protocell translocation across a lipid bi-layer, such as a cellular membrane or endosome lipid bi-layer and is optionally crosslinked onto a lipid bi-layer surface of the protocells. Endosomolytic peptides are a sub-species of fusogenic peptides as described herein. In both the multilamellar and single layer protocell embodiments, the non-endosomolytic fusogenic peptides (e.g., electrostatic cell penetrating peptide such as R8 octaarginine) are incorporated onto the protocells at the surface of the protocell in order to facilitate the introduction of protocells into targeted cells (APCs) to effect an intended result (to instill an immunogenic and/or therapeutic response as described herein). The endosomolytic peptides (often referred to in the art as a subset of fusogenic peptides) may be incorporated in the surface lipid bi-layer of the protocell or in a lipid sublayer of the multilamellar protocell in order to facilitate or assist in the escape of the protocell from endosomal bodies. Representative electrostatic cell penetration (fusogenic) peptides for use in protocells include an 8 mer polyarginine (H₂N-RRRRRRRR-COOH, SEQ ID NO:1), among others known in the art, which are included in protocells in order to enhance the penetration of the protocell into cells. Representative endosomolytic fusogenic peptides (“endosomolytic peptides) include H5WYG peptide, H₂N-GLFHAIAHFIHGGWHGLIHGWYGGC-COOH (SEQ ID. NO: 1), RALA peptide (NH₂-WEARLARALARALARHLARALARALRAGEA-COOH, SEQ ID NO: 8), KALA peptide (NH₂-WEAKLAKALAKALAKHLAKALAKALKAGEA-COOH), SEQ ID. NO:9), GALA (NH2-WEAALAEALAEALAEHLAEALAEALEALAA-COON, SEQ ID NO:10) and INF7 (NH2-GLFEAIEGFIENGWEGMIDGWYG-COOH, SEQ ID. NO:11), among others. At least one endosomolytic peptide is included in protocells in combination with a viral antigen (often pre-ubiquitinylated) and/or a viral plasmid (which expresses viral protein or antigen) in order to produce CD8+ cytotoxic T cells pursuant to a MHC class I pathway,

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides and other functional moieties (for example toll receptor agonists for immunogenic) to the phospholipid bi-layer, to link nuclear localization sequences to histone proteins for packaging supercoiled plasmid DNA and in certain instances, to crosslink lipids in the lipid bi-layer of the protocells. There are a large number of crosslinking agents which may be used in many commercially available or available in the literature. Exemplary crosslinking agents for use, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMFH), NHS-(PEG)ₙ-maleimide, succinimidyl-[(N-maleimidopropionamido)-tetracosaethyleneglycol] ester (SIVI(PEG)₂₄), and succinimidyl 6-[3′-(2-pyridyldithio)-propionamido] hexanoate (LC-SPDP), among others.

The term “toll-like receptor (TLR) agonist” includes but is not limited Pam3Cys,

HMGB1, Porins, HSP, GLP (agonists for TLR1/2); BCG-CWS, HP-NAP, Zymosan, MALP2, PSK (agonists for TLR 2/6); dsRNA, Poly AU, Poly ICLC, Poly I:C (agonists for TLR3); LPS, EDA, HSP, Fibrinogen, Monophosphoryl Lipid A (MPLA) (agonists for TLR4); Flagellin (agonist for TLR5); Imiquimod (agonist for TLR7): and ssRNA, PolyG10 and CpG (agonists for TLR8), as described by Kaczanowka et al., 2013. TLR agonists may be covalently linked to components of the lipid bi-layer using conventional chemistry as described herein above for the fusogenic peptides.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions, alone or in combination with another agent. It is noted that prophylaxis will rarely be 100% effective; consequently the terms prevention and reducing the likelihood are used to denote the fact that within a given population of patients or subjects, administration with compounds will reduce the likelihood or inhibit a particular condition or disease state (in particular, the worsening of a disease state such as the growth or metastasis of cancer) or other accepted indicators of disease progression from occurring. The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments which include a MET binding peptide complexed to the protocell.

The term “anti-cancer agent” is used to describe a compound which can be formulated in combination with one or more compositions comprising protocells and optionally, to treat any type of cancer, in particular hepatocellular or cervical cancer, among numerous others. Anti-cancer compounds which can be formulated with compounds include, for example, Exemplary anti-cancer agents which may be used include, everolimus, trabectedin, abraxane, ILK 286, AV-299, DN-101 , pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a P13 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticillimumab, ipillimumab, gossypol, Bio 111 , 131-I-TM-601 , ALT-110, BIO 140, CC 8490, cilengitide, gimatecan, IL13-PE38QQR, INO 1001 ,IPdR₁ KRX-0402, lucanthone, LY 317615, neuradiab, vitespen, Rta 744, Sdx 102, talampanel, atrasentan, Xr 311 , romidepsin, ADS-100380, sunitinib, 5-fluorouracil, vorinostat, etoposide, gemcitabine, doxorubicin, liposomal doxorubicin, 5′-deoxy-5-fluorouridine, vincristine. temozolomide, ZK-304709, seliciclib; PD0325901 , AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate salt of [D-Ser(But)6,Azgly10] (pyro-Glu-His-Trp-Ser-Tyr-D-Ser(But)-Leu-Arg-Pro-Azgly-NH₂ acetate [C₅₉H₈₄N₁₈O₁₄-(C₂H₄O₂)x where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714, TAK-165, HKI-272, erlotinib, iapatinib, canertinib, ABX-EGF antibody, erbitux, EKB-569. PKI-166, GW-572016, lonafamib, BMS-214662, tipifamib, amifostine, NVP-LAQ824, suberoyl anilide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291 , squalamine, endostatin, SU5416, SU6668, EMD121974, interleukin-12, IM862, angiostatin, vitaxin, droloxifene, idoxifene, spironolactone, finasteride, cimetidine, trastuzumab, denileukin diftitox, gefitinib, bortezomib, paclitaxel, cremophor-free paclitaxel, docetaxel, epithilone B, BMS-247550, EMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001 ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidoniide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

### Exemplary Nanoparticles and Lipid Encapsulated Nanoparticles (Protocells)

In certain embodiments, nanoparticles and protocells generally range in size from greater than about 8-10 nm to about 5 μm in diameter, e.g., about 20-nm-3 μm in diameter, about 10 nm to about 500 nm, about 20-200-nm (including about 150 nm, which may be a mean or median diameter), about 50 nm to about 150 nm, about 75 to about 130 nm, or about 75 to about 100 nm. As discussed above, the protocell population is considered monodisperse based upon the mean or median diameter of the population of protocells. Size is important to therapeutic and diagnostic aspects as particles smaller than about 8-nm diameter are excreted through kidneys, and those particles larger than about 200 nm are often trapped by the liver and spleen. Thus, in once embodiment, monosized protocells are provided of less than about 150 nm for drug delivery and diagnostics in the patient or subject.

In certain embodiments, protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Exemplary pore sizes of mesopores range from about 2-30 nm; they can be monosized or bimodal or graded—they can be ordered or disordered (essentially randomly disposed or worm-like).

Mesopores (IUPAC definition 2-50-nm in diameter) may be ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g., if a templating moiety in the aerosol process is not used. They could also be enlarged to macropores, e.g., 50-nm in diameter.

Pore surface chemistry of the nanoparticle material can he very diverse—all organosilanes yielding cationic, anionic, hydrophilic, hydrophobic, reactive groups—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions.

In some embodiments, the lipid bi-layer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bi-layers, can vary widely in molecular weight (although PEG ranging from about 10 to about 100 units of ethylene glycol, about 15 to about 50 units, about 40 to 50 units, about 15 to about 20 units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bi-layer.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE),1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid bi-layer of protocells according to an embodiment. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In certain embodiments, the lipid bi-layer is comprised of one or more phosphatidyl-cholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

In other embodiments: (a) the lipid bi-layer is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]: and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid bi-layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inosito1-5-phosphate (LPI-5-P), lysophosphatidyl-inosito1-3,4-diphosphate (LPI-3,4-P2), lysophosphatidyl-inositol-3,5-diphosphate (LPI-3,5-P2), lysophosphatidyl-inosito1-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI),

In still other illustrative embodiments, the lipid hi-layer is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG),

In still other embodiments, the lipid bi-layer comprises one or more PEG-containing phospholipids, for example 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-FEG), 1,2-distearoyl-sn-glycero-3-phosphoethanolamine-N-[amino(polyethylene glycol)] (DSPE-PEG-NH₂) (DSPE-PEG). In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N[amino(polyethylene glycol)-2000] (DSPE-PEG₂₀₀₀-NH₂) which can be used to covalent bind a functional moiety to the lipid bi-layer.

In some embodiments, the release profile of cargo components in protocells can be more controllable as compared with when only using liposomes as known in the prior art. The cargo release can be determined by, for example, interactions between the porous core and the lipid bi-layer and/or other parameters such as pH value of the system. For example, the release of cargo can be achieved through the lipid hi-layer, through dissolution of the porous silica; while the release of the cargo from the protocells can he pH-dependent.

In certain embodiments, the pH value for cargo is often less than 7, or about 4,5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

### Formulations and Administration

Generally, dosages and routes of administration of the nanoparticles or protocells are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the nanoparticles or protocells may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 95%, about 0.25% to about 85%, about 0.5% to about 75% by weight of a compound/composition or compounds/compositions, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers,

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or would be apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection (including cancer and) comprise administration an effective amount of a pharmaceutical composition comprising therapeutic protocells and optionally at least one additional bioactive (e.g., anti-viral) agent.

Diagnostic methods may comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer.

In one embodiment, the pores in the MHySNs (nanoparticles) are at least 25 nm in diameter. In one embodiment, the pores are less than about 20 nm in diameter, or about 10 to about 20 nm in diameter or about 5 to about 10 nm in diameter or about 5 to about 15 nm in diameter. In one embodiment, the nanoparticles or lipid containing nanoparficles are about 125 nm to about 350 nm in diameter. In one embodiment, the nanoparticles or lipid containing nanoparticles are about 125 nm to about 250 nm in diameter. In one embodiment, the nanoparticles or lipid containing nanoparticles are about 200 nm to about 350 nm in diameter. In one embodiment, the nanoparticles or lipid containing nanoparticles are about 250 nm to about 400 nm in diameter. In one embodiment, the nanoparticles or lipid containing nanoparticles are about 450 nm to about 600 nm in diameter. In one embodiment, the nanoparticles or lipid containing nanoparticles are about 500 nm to about 900 nm in diameter.

In one embodiment, the lipid layer comprises DOTAP, cholesterol, DSPE, DSPC, DPPC, or any combination hereof. In one embodiment, the lipid layer has about 55% to 65%, 65% to 75%, or 75% to 82% DPPC, about 12% to about 16% 16% to about 21% or about 22% to about 26% mole percent DOTAP, about 3% to about 8%, about 8% to about 15% or 10% to about 20% mole percent cholesterol, about 1% to about 5%, or about 5% to about 10% mole percent DSPE PEG, or any combination thereof. In one embodiment, the formulation comprises DPPC, cholesterol, DOTAP and DSPE.

In one embodiment, the MHySNs have a diameter of about 10 nm to about 100 nm, a pore size of about 5 nm to about 15 nm, and may be enveloped by a lipid bi-layer comprising DPPC, cholesterol, DOTAP and DSPE in mol ratios of about 6-80 DPPC, 10 to 25 of DOTAP, 1 to 12 of cholesterol and 1 to 15 of DSPE PEG. In one embodiment, the MHySNs have a diameter of about 10 nm to abuot 75 nm, a pore size of about 5 nm to about 15 nm, and may be enveloped by a lipid bi-layer comprising DPPC, cholesterol, DOTAP and DSPE in mol ratios of about 6-80 DPPC, 10 to 25 of DOTAP, 1 to 12 of cholesterol and 1 to 15 of DSPE PEG. In one embodiment, the lipid bi-layer has 65 to 75% mol % DPPC, 15 to 25 mol % DOTAP, 5 to 8 mol % cholesterol, and 1 to 5 mol 9/0 DSPE PEG. In one embodiment, the lipid bi-layer has 60 to 70% mol % DPPC, 10 to 20 mol % DOTAP, 8 to 12 mol % cholesterol, and 5 to 15 mol % DSPE PEG. In one embodiment, the lipid bi-layer has 65 to 75% mol % DPPC, 15 to 25 mol % DOTAP, 2 to 6 find % cholesterol, and 0.5 to 4 mol % DSPE PEG.

In one embodiment, the MHySNs have a diameter of about 100 nm to about 200 nm, a pore size of about 5 nm to about 15 nm, and may he enveloped by a lipid bi-layer comprising DPPC, cholesterol, DOTAP and DSPE in mol ratios of about 6-80 DPPC, 10 to 25 of DOTAP, 1 to 12 of cholesterol and 1 to 15 of DSPE PEG. In one embodiment, the lipid bi-layer has 65 to 75% mol % DPPC, 15 to 25 mol % DOTAP, 5 to 8 mol % cholesterol, and 1 to 5 mol % DSPE PEG. In one embodiment, the lipid bi-layer has 60 to 70% mol % DPPC, 10 to 20 mol % DOTAP, 8 to 12 mol % cholesterol, and 5 to 15 mol % DSPE PEG. In one embodiment, the lipid bi-layer has 65 to 75% mol % DPPC, 15 to 25 mol % DOTAP, 2 to 6 mol % cholesterol, and 0.5 to 4 mol % DSPE PEG.

In one embodiment, the MHySNs have a diameter of about 200 nm to about 350 nm, a pore size of about 5 nm to about 15 nm, and may he enveloped by a lipid bi-layer comprising DPPC, cholesterol, DOTAP and DSPE in mol ratios of about 6-80 DPPC, 10 to 25 of DOTAP, 1 to 12 of cholesterol and 1 to 15 of DSPE PEG. In one embodiment, the lipid hi-layer has 65 to 75% mol % DPPC, 15 to 25 mol % DOTAP, 5 to 8 mol % cholesterol, and 1 to 5 mol % DSPE PEG. In one embodiment, the lipid bi-layer has 60 to 70% mol % DPPC, 10 to 20 mol % DOTAP, 8 to 12 mol % cholesterol, and 5 to 15 mol % DSPE PEG. In one embodiment, the lipid bi-layer has 65 to 75% mol % DPPC, 15 to 25 mol % DOTAP, 2 to 6 mol % cholesterol, and 0.5 to 4 mol % DSPE PEG.

In one embodiment, the MHySNs have a diameter of about 350 nm to about 600 nm, a pore size of about 5 nm to about 15 nm, and may be enveloped by a lipid bi-layer comprising DPPC, cholesterol, DOTAP and DSPE in mol ratios of about 6-80 DPPC, 10 to 25 of DOTAP, 1 to 12 of cholesterol and 1 to 15 of DSPE PEG. In one embodiment, the lipid hi-layer has 65 to 75% mol % DPPC, 15 to 25 mol % DOTAP, 5 to 8 mol % cholesterol, and 1 to 5 mol % DSPE PEG. In one embodiment, the lipid bi-layer has 60 to 70% mol % DPPC, 10 to 20 mol % DOTAP, 8 to 12 mol % cholesterol, and 5 to 15 mol % DSPE PEG. In one embodiment, the lipid bi-layer has 65 to 75% mol % DPPC, 15 to 25 mol % DOTAP, 2 to 6 mol % cholesterol, and 0.5 to 4 mol % DSPE PEG.

### Bioactive Agents for Nanoparticles and Protocells and Uses Thereof

Doxorubicin is an anthracycline anticancer drug known for its immunogenic capabilities. In addition to its cytotoxic nature, it benefits nanotechnology studies based on innate fluorescence, the latter beneficial for imaging. It is regarded as an ICD-inducing drug due to its ability to elicit the translocation of calreticulin and release of ATP and high mobility group protein B1 (HMGB1), the latter a Toll-like receptor 4 (TLR-4) ligand, activating DCs and T-cells.

Beyond antracyclines, platinum-based drugs have long been studied for their antitumor properties, yet not all are immunogenic. Cisplatin was the first and is one of the most potent platinum-based anti-tumor compounds. It was approved by the Food and Drug Administration (FDA) in 1978. Cisplatin, also known as cis-diamminedichloroplatinum (II), is inert in nature due to its d8 low-spin electron configurations resulting in high crystal field splitting energy. This compound has proven to be a very potent anticancer agent and has applications for the treatment of several types of cancer including ovarian, prostate, bladder, cervical and many more. The advent of other platinum-based chemotherapeutics has been driven by efforts to reduce side effects and drug resistance associated with cisplatin-based therapy. Like cisplatin, oxaliplatin is a square planar inorganic platinum metal centered compound with a +II oxidation state. Both complexes operate under similar mechanisms through forming DNA adducts. Cisplatin, however, has been shown to form DNA adducts 10 times better than oxaliplatin. Cisplatin works by first entering the cell, and then undergoing ligand exchange between the two chloride ions with two molecules of water. Thus, the drug is activated and is subsequently capable of binding with DNA, specifically the N7 position of guanine. This essentially results in DNA damage, which prevents the DNA from replicating, thereby inhibiting the ability of the cancer cells to multiply. Conversely, this activates a cellular DNA damage response that aids in the recovery of healthy cells. Oxaliplatin is thought to operate via a very similar pathway, though this has not been entirely elucidated as of yet. Platinum-based drugs have shown higher capacities than other chemotherapeutics to improve T-cell activation by DCs. Both cisplatin and oxaliplatin are able to trigger the active release of ATP and HMGB1, two of the three requirements for ICD. Surprisingly, while oxaliplatin is able to support calreticulin migration, cisplatin is not considered immunogenic due to its inability to expose calreticulin on the cell surface.

Emerging data suggest that the co-delivery of cisplatin with other anticancer agents improves drug resistance. Although not officially considered an immunogenic cell death inducer, when paired with radiotherapy, cisplatin is capable of eliciting full ICD. It has additionally been proven that both γ-radiation or UVC radiation alone have been successful in stimulating the exposure of calreticulin. Both photosensitizers and additional anticancer agents including other chemotherapeutics have proven to reduce drug resistance when conjugated with cisplatin. In summary, chemotherapeutic drugs have the potential to induce a dual therapeutic effect, death of highly replicating cancer cells and stimulation of anti-cancer immune responses. With respect to immunotherapy, nanoparticles provide an opportunity to co-deliver chemotherapeutics and other adjuvants that enhance the immune priming potential of frontline chemotherapy and radiation, helping to alleviate immune suppression in the tumor microenvironment and enhancing immunogenicity.

Microbial products, known as pathogen-associated molecular patterns (PAMPs), stimulate AFC maturation through TLR or other pattern recognition receptors (PRRs), promoting expression of co-stimulatory molecules and cytokines.

As described below, immunogenic mesoporous hybrid siliceous nanoparticles (MHySN) were prepared for logic-embedded, sequential presentation of TLR-4 ligand, followed by ICD-inducing DOX. MHySN are formed by liposome fusion on high surface area (>500 m²/g) mesoporous siliceous nanopailicle (NP) cores. Monophosphoryl lipid-A (MPL), a nontoxic derivative of lipopolysaccharide (LPS), is a T helper (Th)1-biased adjuvant that binds to surface/endosomal TLR-4 on APC and activates a proinflammatory signaling cascade (Dasari & Tchounwou, 2014). Integration of MPL (or other PAMP, DAMP, cytokine or other immune stimulant) into the supported lipid bilayer confers the ability to both target and activate ARC, polarizing macrophages, dendritic and natural killer cells to a Th1 phenotype.

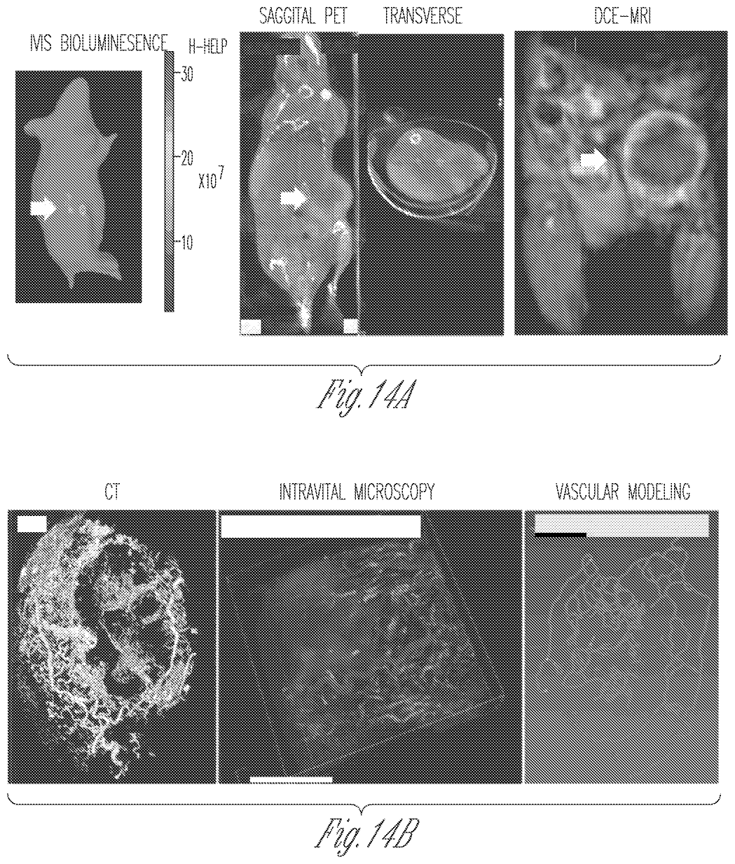

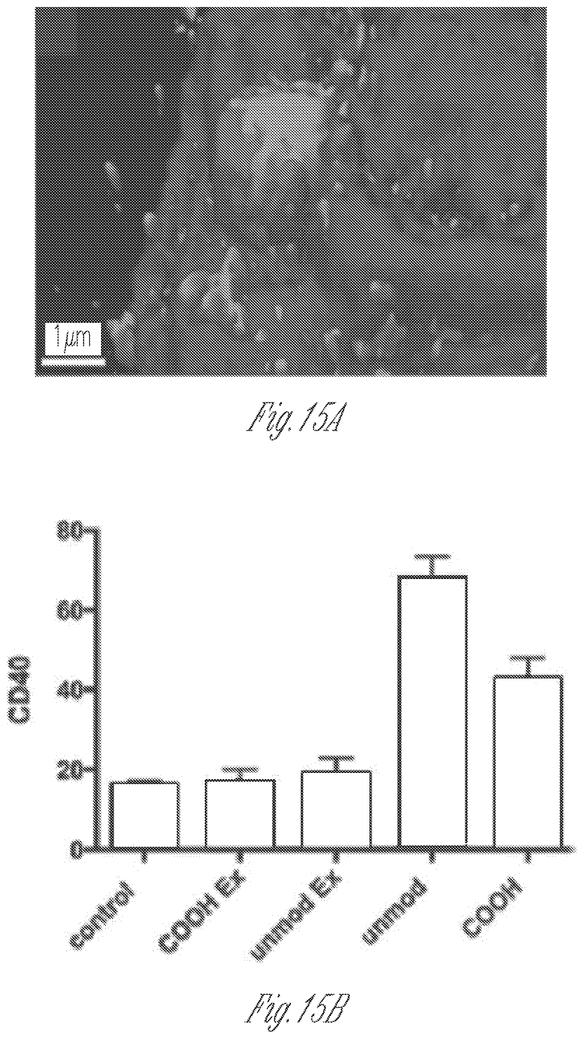

Nanoparticles (NPs) for multivalent presentation of adjuvant represent an emergent field with evidence for enhanced activation of immune cells over free adjuvants. Liposome presentation of the Toll-like receptor (TLR)-4 ligand monophosphoryl lipid (MPL)-A and interleukin (IL)-12 (Nano-MPL-12) synergistically activate antigen presenting cells (APC), block tumor growth (FIG. 12) and enhance migration of dendritic cells (DC) to the draining lymph node for activation of cancer-specific cytolytic T cells. Further, mesoporous hybrid siliceous NPs (MHySN) with supported MPL-lipid bilayers activate DC and stimulate antigen processing and presentation.

Anthracyclines, such as doxorubicin (DOX), increase monocyte and DC infiltration into tumors and trigger immunogenic cell death (ICD), sensitizing tumors to TLR-4-dependent CD8⁺ T cell-mediated immune attack. Addition of a TLR-9 ligand, such as CpG oligodeoxynucleotide (ODN), stimulates IL-12 secretion, essential for control of metastases, and works synergistically with TLR-4 ligand to elicit anti-tumor responses. MHySN has a high capacity for drug loading, with retention and release under the influence of the chosen siliceous core and supported lipid bilayer. The present disclosure provides for MHySN for logic-embedded sequential presentation of MPL in the supported lipid bilayer, followed by pH-triggered release of CpG ODN and DOX to stimulate antitumor immunity, with sustained immunity achieved in vivo using concurrent administration of anti-PD-1 antibody for immune checkpoint inhibition.

A positive therapeutic effect in mice was observed with parental 4T1 tumor when combining immunogenic liposomes with anti-PD-1 antibody, however, mice with lentiviral-transduced 4T1 tumors experienced immune-related adverse events (irAEs), similar to some patients. Biological profiles that accompany irAEs were observed to identify imaging and molecular markers that predict irAEs in patients. Dynamic contrast-enhanced magnetic resonance imaging (DCE-MRI) can be used to image changes in tumor vasculature, positron emission tomography (PET) with ¹⁸F-fluorodeoxyglucose (FDG) to enable imaging of altered metabolic rates, and flow cytometry, fluorescent microscopy and Luminex technology will be used to profile the cellular and cytokine milieu and activation markers in tumor, lymphatic tissues and serum. Single photon emission computed tomography (SPECT) and PET co-registered with computed tomography (CT) is used to image MHySN pharmacokinetics, biodistribution, and co-localization of adoptively-transferred macrophages and MHySN.

In one embodiment, the nanoparticles benefit from: a) immunogenic presentation of antigen arising from DOX-induced apoptosis; b) stimulation of dual TLR signaling pathways in phagocytic antigen presenting cells (ARC); and/or c) inhibition of checkpoint regulators that would otherwise silence the immune response. Microbial products, known as pathogen-associated molecular patterns (PAMPs), stimulate APC maturation through TLR or other pattern recognition receptors (PRRs), promoting expression of co-stimulatory molecules and cytokines. Immunogenic mesoporous hybrid siliceous nanoparticles (MHySN) for logic-embedded, sequential presentation of TLR-4 ligand, followed by TLR-9 ligand and ICD-inducing DOX were prepared. MHySN are formed by liposome fusion on high surface area (>500 m²/g) mesoporous siliceous nanopailicle (NP) cores. Monophosphoryl lipid-A (MPL), a nontoxic derivative of lipopolysaccharide (LPS), is a T helper (Th)1-biased adjuvant that binds to surface/endosomal TLR-4 on APC and activates a proinflammatory signaling cascade. Integration of MPL into the supported lipid bilayer confers the ability to both target and activate APC, polarizing macrophages, dendritic and natural killer cells to a Th1 phenotype. Unmethylated bacterial CpG oligonucleotide (ODN), co-loaded into the mesoporous core with DOX, binds to TLR-9 present within the endolysosomal compartment, triggering signaling cascades that further stimulate a proinflammatory response. The FDA-approved DOX liposome formulation Doxil® has been shown to reduce cardiotoxicity compared to free DOX, allowing a larger cumulative dose for patients. However, the extended circulation time of polyethylene glycol (PEG)ylated

Doxil® causes dose-limited mucocutaneous toxicities. In one embodiment, MHySN tethers DOX to the siliceous core using either a benzene-bridged silsesquioxane or pH-sensitive linkers. The impact of MPL presentation as compared to PEG on the supported lipid bilayer on biodistribution will be explored in immune competent mice with 4T1 breast tumors. In light of the expected increase in uptake of MHySN by TLR4 expressing APC, we will also explore the role of macrophages in Trojan horse style delivery of MHySN to the tumor using adoptively transferred macrophages preloaded with MHySN.

Proinflammatory Th1 cytokine IL-12 has been introduced into immunogenic cationic MPL-liposomes to enhance antitumor immunity. Here, a less toxic alternative is employed, that is, incorporation of the TLR-9 ligand CpG ODN into the MHySN formulation to induce endogenous secretion of IL-12 and interferon (IFN)-γ. Combined activation of multiple TLR signaling pathways has been demonstrated to be far superior to single TLR ligand presentation for activating macrophages and supporting antitumor immunity in mice. Bacterial DNA stimulates mammalian immune cells based on the presence of unmethylated CpG oligodeoxynucleotides (ODN) in specific sequence contexts. These DNA motifs consist of unmethylated CpG flanked by two 5′ purines and two 3′ pyrimidines. While free CpG ODN suffers rapid elimination and low access to immune cells, NP presentation is multivalent, with abundant exposure to APC. CpG ODN 1826 was used as an exemplary molecule based on demonstrated strong immunostimulatory effects on mouse immune cells and its ability to enhance sensitivity to chemotherapy. Beyond the reduced potential for adverse side effects, CpG ODN based on its small size and its high charge is easier to load in MHySNs and is more cost-effective.

Dramatic preclinical synergy has been demonstrated between tumor vaccines and inhibition of immune checkpoints. One major negative regulator of T cell function is the immune checkpoint molecule PD-1 (programmed cell death protein-1; CD279), expressed on activated T cells. Binding of PD-1 by B7-H1 (PD-L1) on tumor cells suppresses T cell activation. Surface expression of tumor-associated PD-L1 is upregulated following engagement of CD8⁺ T cells with the MHC-antigen complexes on the cancer cell, leading to progressive loss of T cell function. Early clinical data shows that blocking PD-L1/PD-1 interactions with antibodies has a greater than 50% overall response rate in advanced melanoma, unfortunately, a large number of patients also stiffer from immune-related adverse events (irAEs). Interestingly, preliminary studies revealed irAEs, characterized by tumor growth, weight loss and high rates of mortality in response to hamster anti-PD-1 antibody purified from hybridoma clone C1-G4 in mice bearing 4T1 tdTomato red luc (Caliper LifeSciences) tumors but not in mice with 4T1 parental (not shown) or 4T1 luc (Imanis Life Sciences) tumors (FIG. 12). Both of the luciferase-expressing tumors were transduced using lentivirus and sites of integration will be explored using ligation-mediated PCR. SEM and H&E imaging of tumor tissues from control and anti-PD-1 antibody treated mice show high cell density in control tumors and extensive vacuoles in the anti-PD-1 group. Mall et al. (2016) reported that BALB/c mice with 4T1 (gift, origin unknown) tumors undergo high mortality rates in responses to anti-PD-1 antibody (clone J43, hamster IgG) based on xenogenic hypersensitivity reactions (however, isotype control antibody did not elicit similar responses). Conversely, work by Black et al. (2016) showed that recombinant PD-1 confers chemoresistant to DOX and that blocking PD-1 (clone not stated) reduces 4T1 (ATCC) breast cancer metastasis.

As described herein below, MHySN was employed for co-delivery of DOX and TLR ligands. MHySN formulations were selected based on in vitro cell viability, drug release, and activation of APC. The high versatility, loading capacity and layered presentation of components enables production of environmentally sensitive drug carriers.

MHySN (administered free or internalized in adoptively-transferred macrophages) are labeled with ¹¹¹In for in vivo quantitation using SPECT imaging and gamma counting of organs. Co-localization of adoptively transferred HSV1-tk transformed macrophages with MHySN will be evaluated using ¹⁸F-FIAU and PET imaging. Based on high rates of immunogenic MHySN uptake by myeloid cells (both in vivo and in vitro), tumor and lymphatic accumulation of MHySN may exceed that of control PEG-MHySN. And based on the high loading capacity and pH-sensitive release of DOX in MHySN, the MID of MHySN may exceed that reported for Doxil®, and the MID of unloaded MHySN should exceed that of DOX-loaded MHySN.

To evaluate the therapeutic efficacy and immunogenicity of MHySN and anti-PD-1 antibody, variants of the orthotopic syngeneic 4T1 and transgenic MMTV-PyMT mouse models of breast adenocarcinoma are employed. Physiological responses to therapy are monitored through noninvasive imaging with an emphasis on tumor growth, tumor perfusion and metabolic rates using IVIS and calipers, MRI and PET imaging.

Define associated changes in tumor tissue architecture, immunocytes, cytokines, markers of activation, and tissue necrosis using electron and fluorescent microscopy, flow cytometry, cytokine (Luminex) and antibody (ELISA) analysis. MHySN TLR ligand-mediated activation of immunocytes and delivery of pH-triggered release of immunogenic DOX combined with immune checkpoint blockade may ablate tumor cells and stimulate antitumor immunity superior to single-agent MHySN therapy.

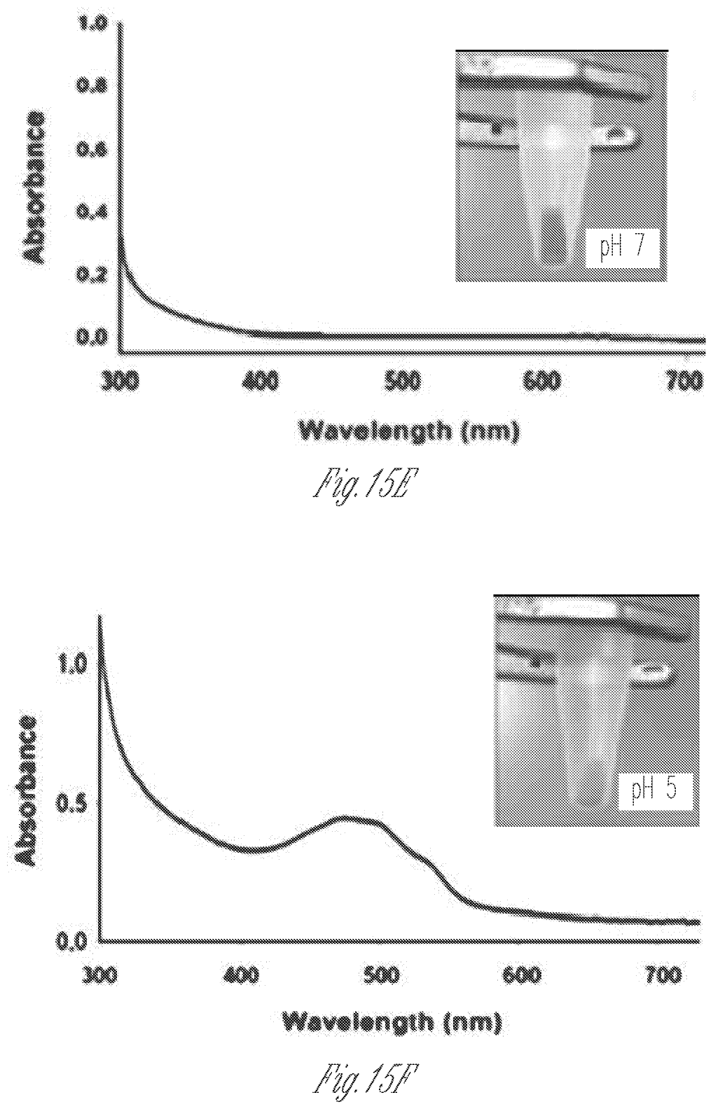

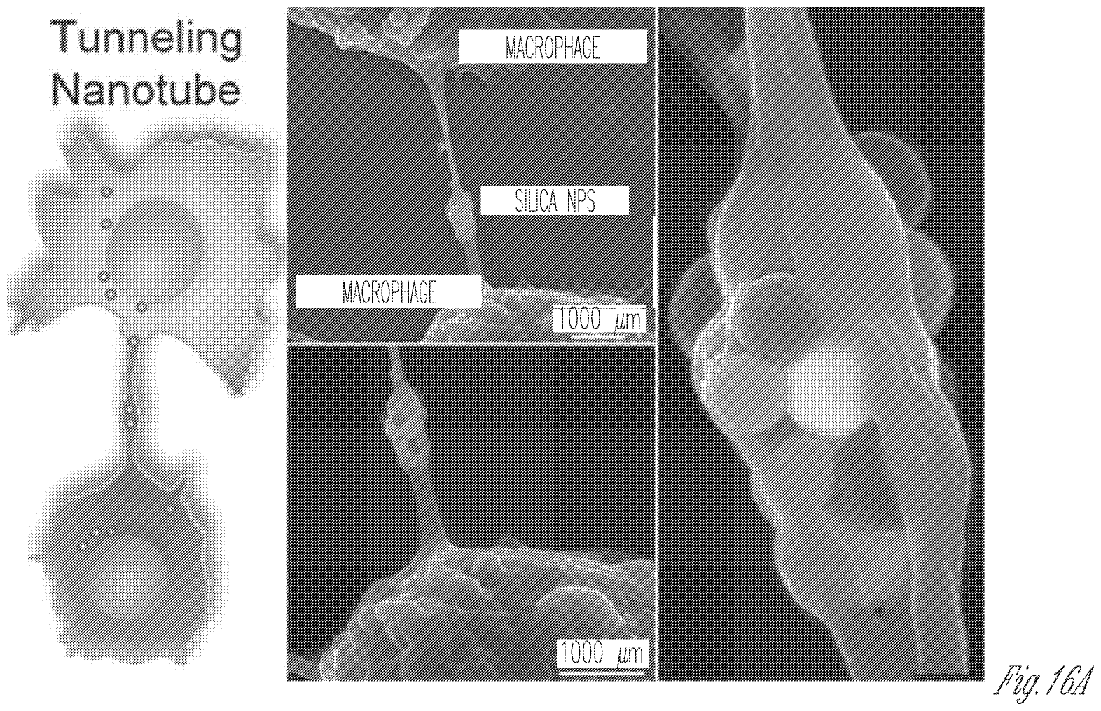

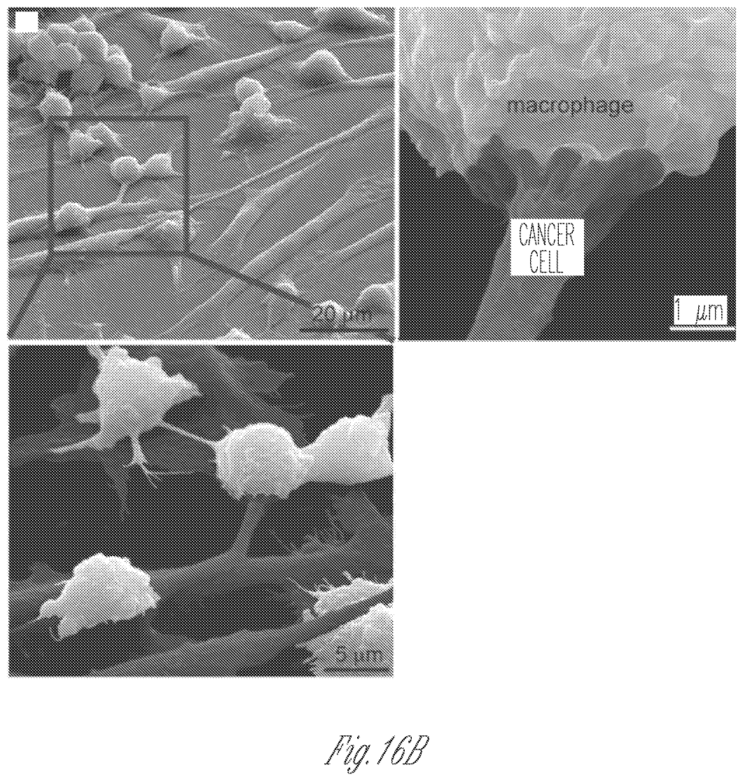

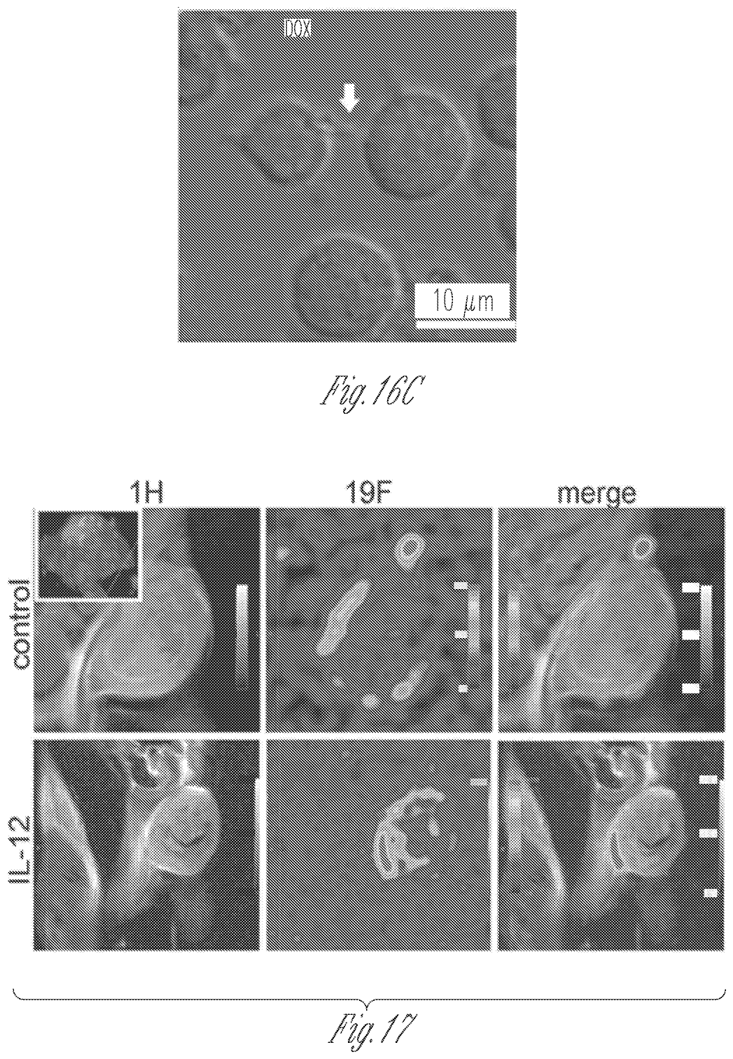

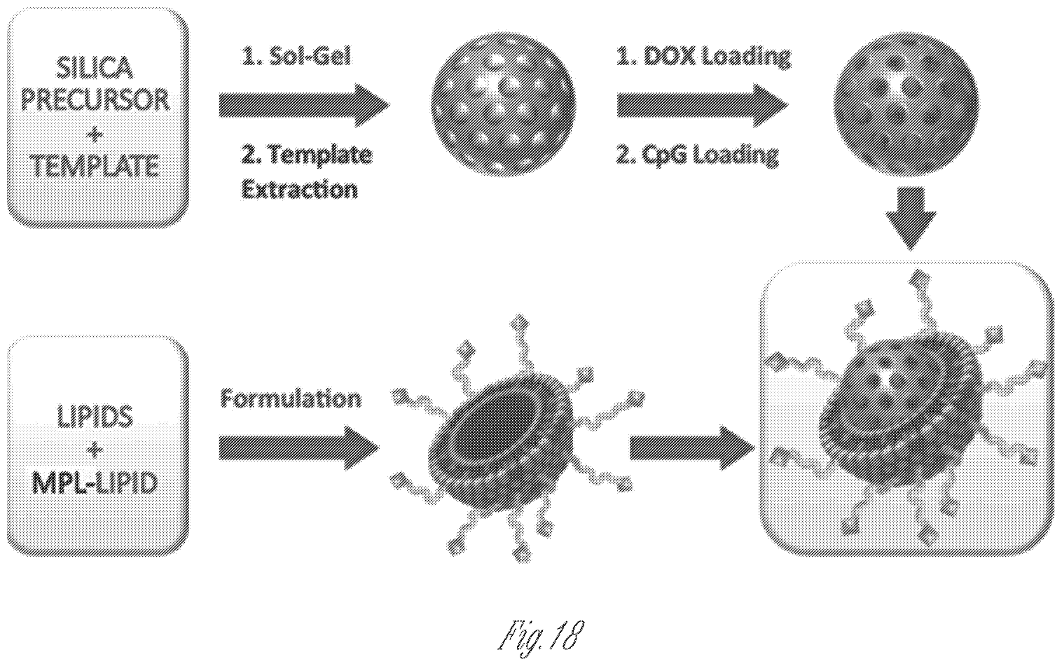

V-sense is a commercial ¹⁹F-perfluorocarbon emulsion used to track macrophage infiltration into inflammatory tissues. Ahrens et al. (2011) successfully quantified V-sense in inflamed tissues of the central nervous system in an ex-vivo model of allergic encephalomyelitis, which they correlated with immunohistochemistry to confirm co-localization of V-sense emulsion droplets and macrophages. Hitchens et al. (2011) detected V-sense labeled macrophages in a model of cardiac allograft rejection. In FIG. 12, the ¹⁹F contrast in tumors is shown based on infiltration of V-sense containing macrophages. In vitro fluorescent imaging confirmed V-sense (red) uptake by RAW macrophages (FIG. 15). Proton and ¹⁹F MR images of microfuge tubes containing variable numbers of RAW cells following incubation with V-sense revealed a positive correlation between cell number and ¹⁹F signal intensity (not shown). FIG. 14 shows ¹⁹F MR images, independently (left) or merged with proton MR images (right), 48 h following intratumoral PBS or IL-12 injection and 12 h following V-Sense injection. The color spectrum indicates the concentration of V-sense, which is analogous to phagocytic myeloid cell density. The intratumoral injection of IL-12 increased the frequency of phagocytic cells localized within the tumor periphery 48 h post-injection compared to PBS injected animals.

Circulating and marginal zone splenic macrophages rapidly engulf nanopailicles. NPs have the ability to polarize macrophages towards an M1 phenotype, and it is well established that blood-derived monocytes can be differentiated towards a DC phenotype. Immune cell activation has a positive impact on trafficking of NPs to both tumor and lymphatic tissue. Macrophages, which can represent up to 70% of the tumor mass, migrate in the blood towards a gradient of chemoattractants present in the tumor. It has been reported that cells in hypoxic environments secrete various chemoattractants that recruit myeloid cells to the hypoxic region. 4T1 tumors quickly become hypoxic and develop necrotic cores, providing a rich foci for macrophage infiltration. Verra et al. (2005). confirmed an increase in tumor infiltration by DC following cytokine immunotherapy, and it was demonstrated that TLR4 ligand-activated DC display enhanced migration to lymphatic tissue, specifically the draining lymph node which is the primary site for early metastatic tumor cell invasion.¹

Two distinct activation states of macrophages exist: 1) conventionally activated M1 macrophages that produce high levels of IL-12, tumor necrosis factor (TNF) and inducible nitric oxide synthase (iNOS); and 2) M2 macrophages, which produce arginase, IL-10, transforming growth factor-β (TGF-β) and prostaglandin E2 (PGE2). M1 macrophages are potent effector cells that kill tumors directly through production of nitric oxide and TNF, and through secretion of Th 1 cytokines. Conversely, M2 macrophages suppress T cell activation and proliferation. Rather than target specific macrophage populations, NPs have the ability to activate and polarize macrophages towards an M1 phenotype.

In one embodiment, macrophages are engineered to express the herpes simplex virus Type 1 thymidine kinase (HSV1-TK) to noninvasively monitor macrophage biodistribution using fluorescent or PET reporters. The MHySN platform is highly efficient at delivering plasmid DNA, and here we will either deliver the pLOX-gfp-iresTK plasmid to splenic macrophages via loading within radiolabeled NPs or create a stable HSV1-TK macrophage cell line using the TK-RFP lentivirus purchased from GenTarget Inc. To study co-localization of ¹¹¹In-NPs and adoptively-transferred carrier myeloid cells, we will coregister SPECT (NP) and PET (cell) images. The positron-emitting isotope ¹²⁴I-FIAU will specifically accumulate in adoptively transferred macrophages based on TK-mediated phosphorylation.

In one embodiment, MSNPs or MHySNs may range in diameter from about 1 nm to about 500 nm, about 5 nm to about 350 nm, about 10 nm to about 300 nm, about 15 nm to about 250 nm, about 20 nm to about 200 nm, about 25 nm to about 350 nm, or about 20 nm to about 100 nm. In one embodiment, the mMSNPs are about 80 to about 100 nm in diameter.

In one embodiment, the lipid bi-layer comprises more than about 50 mole percent an anionic, cationic or zwitterionic phospholipid or said lipid hi-layer comprises lipids selected from the group consisting of 1,2-dioleoyl-sn-glycero-3-phosphocholine (DOPC), 1,2-dipaimitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1 ,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid bi-layer comprises cholesterol. In one embodiment, the lipid bi-layer comprises about 0.1 mole percent to about 25 mole percent of at least one lipid comprising a functional group to which a functional moiety may be complexed via coordinated chemistry or covalently attached. In one embodiment, the lipid comprising a function group is a PEG-containing lipid, optionally wherein said PEG-containing lipid is selected from the group consisting of 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3-phosphoethanolamine wherein said lipid bi-layer comprises more than about 50 mole percent an anionic, cationic or zwitterionic phospholipid or said lipid bi-layer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 FE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid bi-layer comprises cholesterol.

In one embodiment, the MHySNs may induce immunogenic cell death (ICD) as well as interfere in the immunosuppressive indoleamine 2,3-dioxygenase (ICO) pathway. This may be accomplished by conjugating the IDO inhibitor, indoximod (IND), to a lipid bilayer that encapsulates the MHySNs. The porous interior of MHySNs allows for contemporaneous delivery of an ICD-inducing agent, such as oxaliplatin (OX) or doxorubicin. Doxorubicin (DOX) is the classical example of inducing an ICD response, which is characterized by apoptotic cell death, accompanied by the expression of calreticulin (CRT) on dying tumor cell surfaces (Obeid et al., 2007). Oxaliplatin (OX) can also induce an ICD response in various cancer cells, including pancreatic cancer cells (Zhao et al., 2016). CRT provides an “eat-me” signal for dendritic cell (DC) uptake (Obeid et al., 2007; Kraemer et al., 2013). The subsequent release of ATP and a non-histone chromatin protein, high-mobility group box 1 (HMGE-1), from the tumor cells provide adjuvant stimuli to the antigen presenting DC (Kraemer et al., 2013). This cell biological sequence is dependent on the ability of select chemotherapeutic agents, physical stimuli (e.g., irradiation) and cytotoxic viruses to trigger a combination of apoptotic cell death, endoplasmic reticulum stress and autophagy (Apetoh et al., Casares et al., 2006; Fucikova et al., 2011; Michaud et al., 2011; Zappasodi et al., 2010).Thus, ICD chemotherapeutics sensitize tumors to T cell mediated immune responses by triggering immunogenic cell death that is dependent on TLR-4 and CD8⁺ T cells.

The invention will be further described by the following non-limiting examples.

### EXAMPLE 1

Immunogenic cancer cell death (ICD) inducing chemotherapeutics stimulate cancer-specific immunity and alleviate T cell suppression by mechanisms that are dependent on TLR-4 and CD8⁺ T cells. Herein doxorubicin, oxaliplatin or cisplatin-loaded mesoporous silica cores were encapsulated with monophosphoryl lipid (MPL)-A-modified lipid bilayers for co-targeting cancer and antigen presenting cells. Using the ID8 ovarian cancer cell line, cellular uptake and transport phenomenon of mesoporous hybrid silica nanoparticles (MHySN) were studied using flow cytometry and confocal microscopy. Cationic MHySN were rapidly and abundantly internalized by ID8 cancer cells, with localization in endosomes located in the perinuclear region of the cell. The presence of MPL in the lipid supported bilayer enhanced specificity towards antigen presenting cells and supported activation of cells and enhanced antigen processing. In summary, MHySN that co-deliver immunogenic chemotherapy and immune stimulants were prepared and tested, resulting in high levels of cancer cell death and immune cell stimulation, favoring cancer-specific immunity.

### Methods

### Mammalian Cell Culture

ID8 OVA and parental cells were cultured at 37° C. in 5% CO₂, in Dulbecco's Modification of Eagle's Medium acquired from ThermoFisher Scientific to which 50 mL (10%) of Fetal Bovine Serum and 5 mL (1%) Penicillin-Streptomycin antibiotic were added. The bone marrow of female murine C57BL/6 mice was additionally harvested via a 27 g needle. The bone marrow cells were cultured in 6-well plates

Flow Cytometry and Imaging of ID8 Cells with Dox and Cisplatin

Flow Cytometry: 12-well plates were seeded with 1 E5 ID8 cells/well in 2 mL of media (DMEM) with different concentrations of drugs in triplicate. The first plate contained a control, and dosages of 0.1, 0.5, and 2 μg/mL of Doxorubicin. The other well plate contained 0.1, 0.25, 0.5 and 2 μg/mL of cisplatin. Both drug solutions were originally 1.0 mg/mL in water. After the chemotherapeutics were added in sterile conditions (also pipetted up and down after to mix a little) the cells were then prepared for flow after about ˜18 hours. The cell media was removed from the well via a plastic pipette and put in a FACS tube. The wells were washed with 1xPBS which was additionally added to the FACS tube. Then, Trypsin EDTA was added to the wells and then removed in order to release the adherent cells. Thus, once all the cells were harvested, the tubes were centrifuged at 1200 RPM for 4 minutes. Then the supernatant media was poured into a liquid waste container and about 2 mL of 1xPBS was added to the tubes which were centrifuged again at the same settings. Then 100 μL of annexin V buffer was added to the tubes on ice. The fluorescent dye was prepared with 25 μL of Propidium Iodide and 125 μL of Annexin V and 1.10 mL of Annexin V Buffer was added to achieve a final volume of 1,25 mL. 5 μL of this solution was added to each tube and to sit for 15 minutes and after this, 400 μL of annexin buffer was added. Then the sample was run on FACSCalibur using CellQuest™ software (BD Biosciences),

Imaging: 6-well plate were seeded with 2×10⁵ ID8 cells/well on coverslips in 3 mL of media (DMEM). The top three wells all were stained with calreticulin antibody and the bottom three were stained with Propidium Iodide and Annexin V. The first column of wells were both controls, and then other two columns contained a 2 μg/mL dose of Doxorubin and the other two wells contained a 20 μg/mL dose of cisplatin. Both drug solutions were originally 1.0 mg/mL in water but were additionally diluted if necessary. One hour after the addition of chemotherapeutics, the cells were washed, fixed and stained. The media was removed without disturbing the bottom of the well. Then a wash with 1×PBS followe3d by fixation with 2 mL of room temperature paraformaldehyde. After ˜15 minutes, the PFA was removed and the cells were washed with 1×PBS again. Annexin V and Propidium Iodide were diluted to 1.5 mL (500 μL per well, 25 μL/500 μL) or 15 μL of 1:100 dilution of Anti-calreticulin antibody [EPR3924] (Alexa Fluor® 647) were added and incubated for 30 mins. This liquid was then removed and the coverslips were washed with PBS and new PBS was added. The slides were placed on microscope slides and coverslips were mounted with ProLong Gold Nuclear stain with DAPI. The slides were then imaged under a Leica TCS SP8 Confocal Microscope using a 63X/1.4NA oil objective.

### Nanoparticle Synthesis

### Pure Silica Nanoparticles

For a silica starting material, Tetraethyl orthosilicate (TEAS, 0.94 g, 9.02 mmol) was weighed and placed in a round bottomed flask. The reaction stirred and was heated to about 58° C. The surfactant used was Cetyltrimethylammonium chloride (CTAC) water (36 mL, 648.5 mmol), and triethylamine (TEA, 0.1807 g, 1.21 mmol), and cyclohexane solvent (18 mL, 166.6 mmol). The solution contained two immiscible liquids, the bottom with a white aqueous bottom phase and the top, the clear, organic phase. 500 μL of 0.5 mg/mL of RITC dye was additionally added.

### Organosilica Nanoparticles

For organosilica nanoparticle starting materials, 1,4-bis(triethoxysilyl)benzene (0.36 mL, 0.907 mmol) was weighed and placed in a 250 mL beaker with a stir bar. The surfactant used was Cetyltrimethylammonium Bromide (CTAS, 0.2937 g, 18.15 mmol), ammonium hydroxide (135 mL, 43.2 mmol), tetrahydrofuran (THF, 15mL, 184.9 mmol). The beaker was covered with 3 pieces of Parafilm and a circular weight and left to stir at 6 at 60° C. for two hours. 500 μL of 0.5 mg/mL of RITC dye was additionally added.

### Nanoparticle Reaction

After about 6 hours, the stirring was turned off for the pure silica nanoparticles and the beaker was covered with foil. Thus, the organosilica particles stirred overnight with foil covering the flask as well. Both solutions were filtered and poured into ultra-high speed centrifuge tubes. Both centrifuged for 45 minutes at 50,000 RCF. Then they were resuspended in pure ethanol and sonicated. Then they were placed back in the centrifuge at the same settings for 40 minutes. The ethanol supernatant was then removed and the tube was filled about halfway with 1% HCl in EtOH. The pellet was resuspended via sonification for 20 minutes and then centrifuged for 40 minutes. Once again the supernatant was removed and the pellet was resuspended in EtOH and stored in the fridge at 4° C. A pipette was used to remove half of the volume of EtOH and this was replaced by about ¼ of the original volume was replaced with 5% HCl. This solution was then sonicated for 20 minutes and the centrifuged for 40 mins. After removal from the centrifuge, 5 mL of 5% HCl was added and the tubes were sonicated for 20 minutes and centrifuged for another 40. They were resuspended and washed/centrifuged twice more with ethanol. Then the EtOH was removed and the pellets were resuspended in EtOH and filtered with a syringe and put in new centrifuge tubes. Additionally, a known amount was taken to dry and to be used for quantification of concentration.

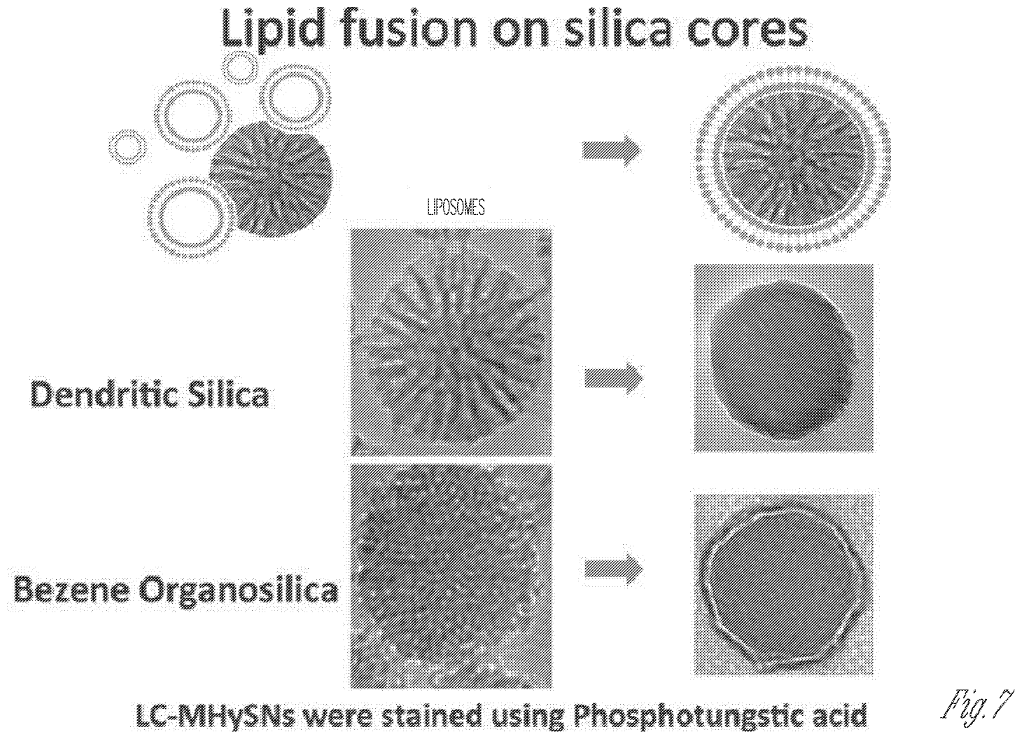

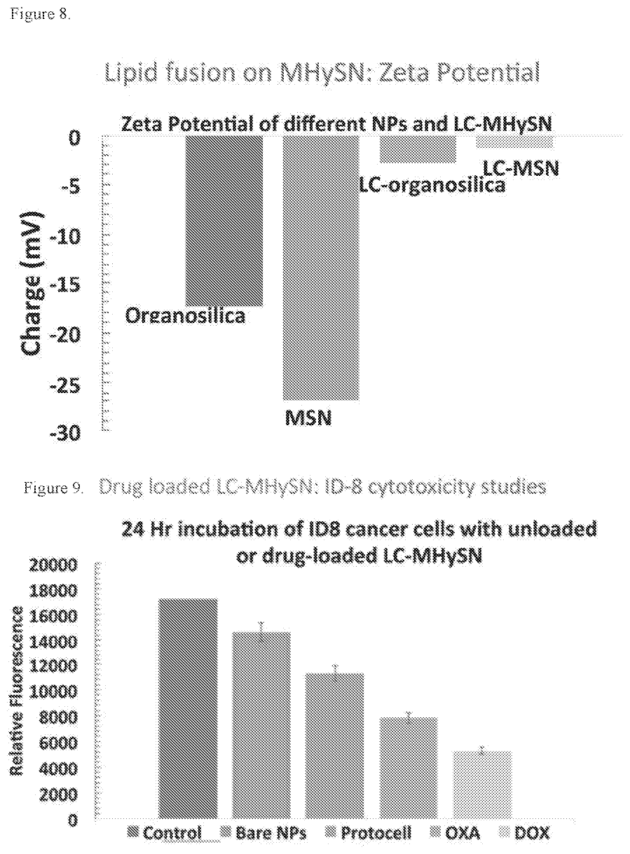

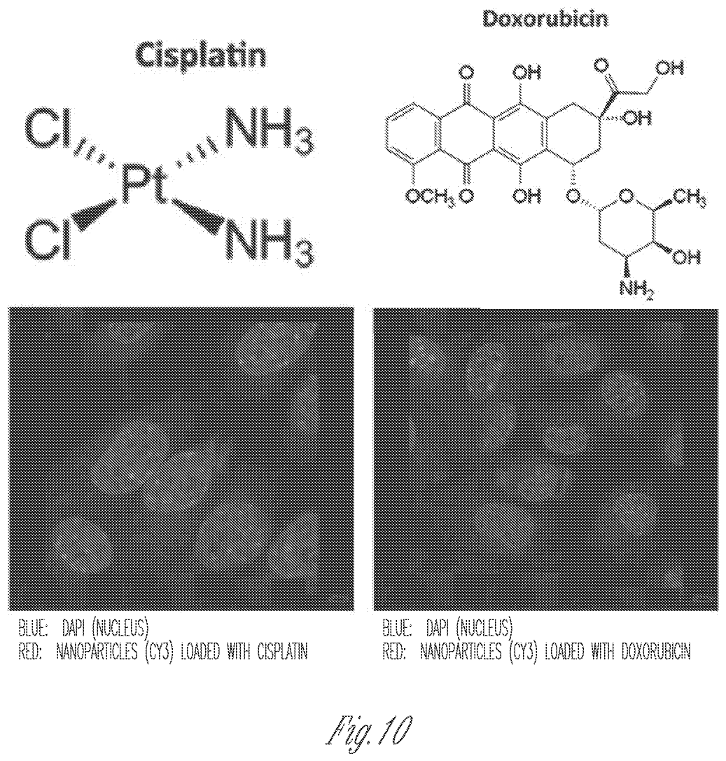

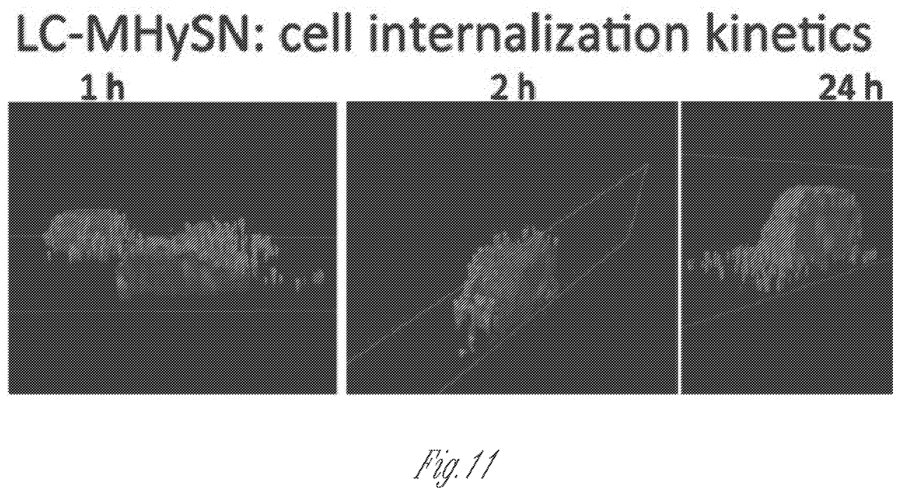

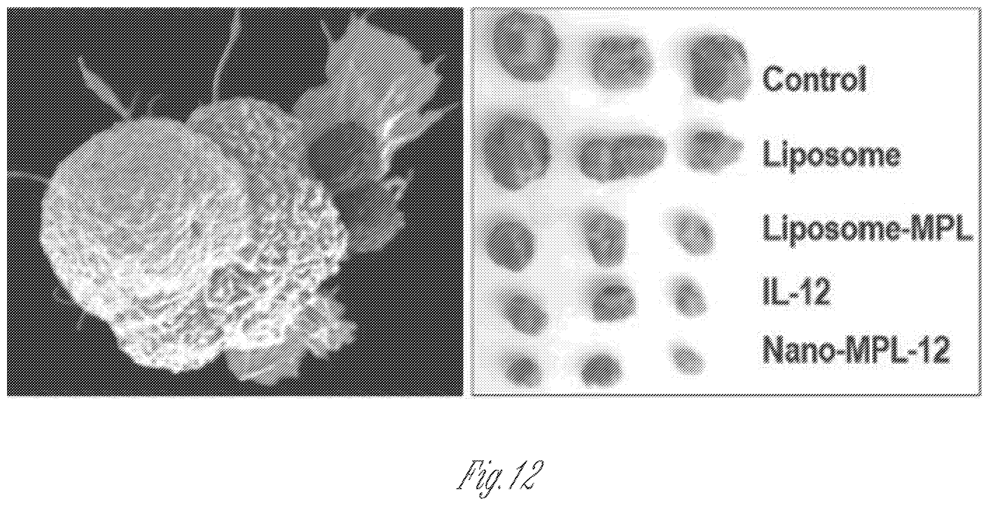

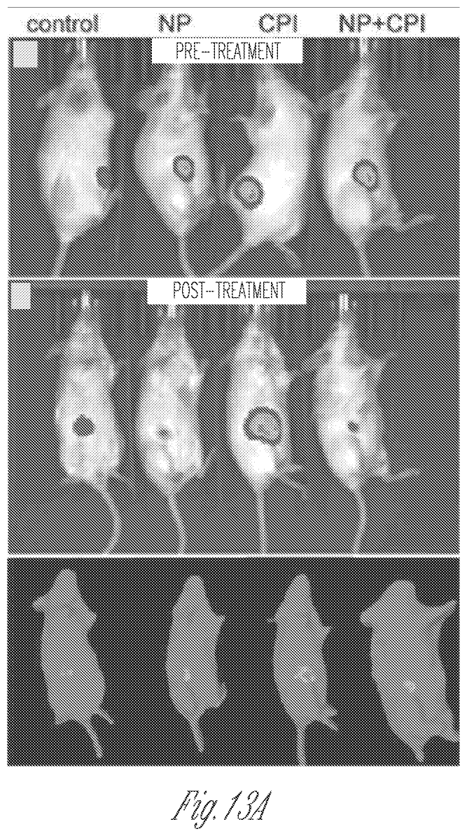

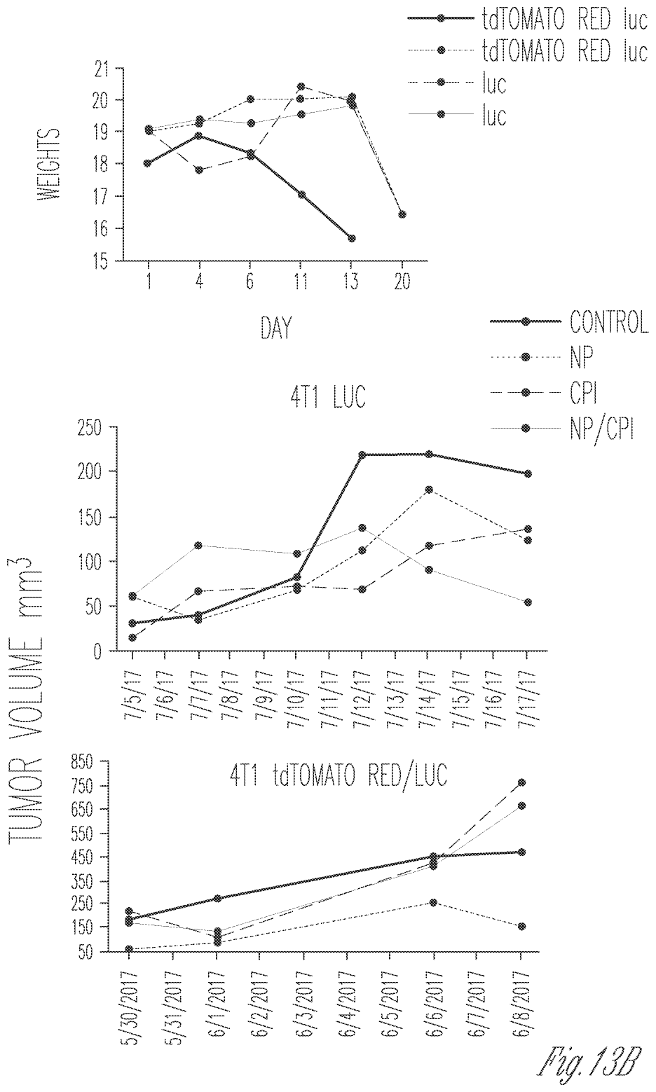

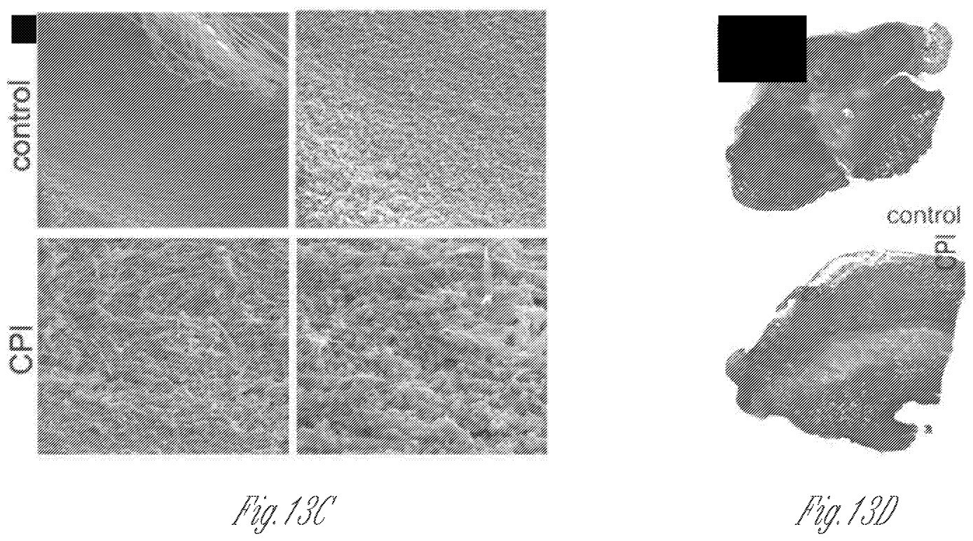

Lipid Formulation The formulations for 3 different liposomes were prepared (see Table 1 in FIG. 5). Each lipid was stored in DMSO in a glovebox and the respective amount of each was added via a micropipette. Once the solution was removed from the glovebox, a rotovap was used to remove the solvent. Then the vial was placed under vacuum overnight. The next day, 1×PBS was to create a solution of liposomes with a 2.5 mg/mL concentration. The solution was sonicated at 40° C. for 45 minutes. Zeta potential and

DLS were run on liposomes and protocells in order to determine the optimal formulation. This was determined by overall size and PdI (Polydispersity Index). The lipid to silica ratio stands at 5:1. Therefore, per 250 μg of NPs were coated with 1250 μL of lipid.

### Drug Loading Capacity:

The determination of the drug loading capacity was essentially determined via UV Spectrometer. The nanoparticles were loaded with 1250 μg of drugs per 250 μg of nanoparticles. The particles were then placed on a rotating wheel overnight to incubate. Known concentrations of doxorubicin and oxaliplatin were both initially measured to determine ne a standard curve. From this equation, the absorbance of the supernatant liquid from the loaded nanoparticles was measured. From this, the loaded mass was determined by subtracting the supernatant value from the original mass of the drug prior to loading. The following equation was thereby utilized:

$$\frac{Mass of Drugs ( µg )}{Mass of NPs ( µg )} * 100$$

Immunogenic Nanoparticle Induced Cell Death via Fluorescence with alamarBlue

48 well plates were prepared by seeding with 0.5×10⁵ ID8 cells in 0.5 mL of media/well. 24 wells were seeded per plate with each variable repeated in triplicate. This included a control, Organosilica Nanoparticles (unloaded), Organosilica NPs with Oxaliplatin and Organosilica NPs with Dox, Bare silica nanoparticles, silica NPs with Oxaliplatin and Silica NPs with Dox. The 48 well plate was similar except for the bare NPs were organosilica in this case. The plates were then incubated for about 24 hours at 37° C. Meanwhile, the loading and preparation of the nanoparticles was emphasized. 50 μg of nanoparticles was prepared for each well. The various drugs and protocells were subsequently washed and placed in cell culture media. The drug solutions were prepared to a concentration of 5 mg/mL and 250 mL (1250 μg) of this was added per 250 μg of NPs. 250 μL of lipid (1250 μg) was also added to the loaded NPs (250 μg) and sonicated in order to form protocells. The addition of 50 μL of alamarBlue (10% per media volume) under sterile conditions to each well as well as media blanks was performed. After one hour of incubation, the samples were run with a BioTek plate reader for both fluorescence and absorbance. The sample was transferred to a black 48 well plate for the fluorescence measurement, while ultimately produced more logical results.

Preparation of Cells for Imaging with Calreticulin Transfection:

### Transfection:

6 well plates were seeded with 2×10⁵ ID8 cells per well. Four wells per plate were utilized; a control, plasmid expressing calreticulin without chemotherapeutics, NPs loaded with oxaliplatin (136 μg) or dox (55.6 μg). All plates were incubated at 37° C. for 1 hour, 2 hours, or 24 hours of exposure to the nanoparticles. The preparation of 3 tubes for the transfection were arranged. The first tube (A) contained 1.5 mL OPTI, 15 μg of plasmid, and 15 μL of reagent. Tube B contained 0.5 μL OPTI, 5 μL PLUS. Tube C contained 1.8 mL of OPTI and 60 μL LTX. The tubes incubated for 10 minutes, then 450 μL of C was added to B. Then A was added to C. These solutions then were left for 30 minutes to further incubate. Then 800 μL was added to each well of A+C and tube B was added to the control. 4 hours later, the chemotherapeutics were added to the respective wells. After two hours of incubation with the drugs, the wells were washed with PBS and fixed in 4% paraformaldehyde.

### Plates for Imaging at Various Timepoints:

6 well plates with coverslips were seeded with 2×10⁵ ID8 cells per well. Four wells per plate were utilized; a control, pure silica nanoparticle protocells with oxaliplatin, pure silica nanoparticle protocells with dox, and bare, unloaded silica nanoparticles. The three plates were utilized for 1 hour, 2 hours, and 24 hours of exposure to the nanoparticles. After the appropriate incubation time with the nanoparticles, the media was removed and the cells were washed twice in 1×PBS. Then 1% BSA with 10% serum was added sat for 10 minutes. Then the block was removed and 0.5 mL of calreticulin antibody was added per slide and allowed to sit for 30 minutes. Then the slides were washed with cold PBS and fixed in cold 4% paraformaldehyde for 15 minutes. After a final PBS wash, the coverslips were mounted to the slides using ProLong Gold Nuclear stain with DAPI. The slides were then imaged under a Leica TCS SP8 Confocal Microscope using a 63X/1.4NA oil objective.

Drug Titration with Oxaliplatin

12 well plates were seeded with 1×10⁵ cells/well with ID8 wild type took place with 2 mLs of media per well. The cells were allowed to incubate for one day. Cells were treated in triplicated with PBS, 40, 50, 60, 70, 80, 90, or 100 μg/mL oxaliplatin. The cells were then prepared for flow cytometry. The media was removed and placed in FACS tubes. The wells were washed with PBS and then trypsin was used to relates the cells wihc were transferred to tubes. Cells were washed wioth PBS and then 400 μL of 1% BSA in PBS were added to each tube. 10 μL of 1 mg/mL PI was added to each sample 10 min before analysis with samples stored on ice.

### TEM Sample Preparation

A suspension of both silica and organosilica protocells in PBS (0.1 mg/mL, 5 μL) was added onto a TEM holey carbon copper grid and kept drying for a few minutes. Phosphotungstic acid (PTA) solution (2% in PBS, 5 μL) was added then quickly removed with a Kimtech® after 10-15 seconds. The grid was then washed by water (15 μL) three times and kept under air for drying.

TEM holey carbon copper grids were washed with 500 μL of EtOH, A suspension of both organosilica nanoparticles in EtOH (13.5 mg/mL, 20 μL) as well as silica (13.5 mg/mL, 10 μL) were added to the grids. Both were left to dry for about 5 minutes and then were placed in the sample grid holder.

### Results

Combined activation of multiple TLR signaling pathways has been demonstrated to be far superior to single TLR ligand presentation for activating macrophages and supporting antitumor immunity in mice (Zhang et al., 2016; Martins et al., 2011; Obeid et al., 2007). Bacterial DNA stimulates mammalian immune cells based on the presence of unmethylated CpG oligodeoxynucleotides (ODN) in specific sequence contexts. These DNA motifs consist of unmethylated CpG flanked by two 5′ purines and two 3′ pyrimidines (Wheeler et al., 2001). While free CpG ODN suffers rapid elimination and low access to immune cells, NP presentation is multivalent, with abundant exposure to APC.

Beyond the reduced potential for adverse side effects, CpG ODN based on its small size and its high charge is easy to load in MHySN. Unmethylated bacterial CpG oligonucleotide (ODN), can be co-loaded into the mesoporous core with DOX (or other ICD chemotherapteutic), binds to TLR-9 present within the endolysosomal compartment, triggering signaling cascades that further stimulate a proinflammatory response. The FDA-approved DOX liposome formulation Doxil® has been shown to reduce cardiotoxicity compared to free DOX, allowing a larger cumulative dose for patients. However, the extended circulation time of polyethylene glycol (PEG)ylated Doxil® causes dose-limited mucocutaneous toxicities (Faivre et al., 2003). MHySN tethers DOX to the siliceous core using either a benzene-bridged silsesquioxane or pH-sensitive linkers. In light of the expected increase in uptake of Following uptake of MHySN, ARC are activated and can function as carriers for delivery of MHySN to cancer cells.

Advantages of theMHySN platform include the well-known assets of liposome formulations (low inherent toxicity, tailored environmentally responsive lipid bilayers) and the high stability and capacity for loading and simultaneous delivery of multiple cargos by porous nanomaterials. As stated, logic-embedded MHySN sequentially and logically presents cargo, beginning with MPL for engagement of TLR-4 on immune cells. Once internalized, the dissociation of the supported lipid bilayer in the acidic endosome exposes or releases TLR-9 ligand (e.g., CpG ODN) that in turn engages receptors within the endolysosomal compartment (Roers et al., 2016; Yotsumoto et al., 2008). Transfer of DOX from immune cells to tumor cells has been proposed to occur by release from dead cells. Choi et al. (Choi et al., 2012) demonstrated that systemically-injected peritoneal macrophages loaded with liposomal DOX successfully migrate to tumors and inhibit tumor growth. Furthermore, Soma et al. (Soma et al., 2000) showed that DOX-induced secretion of nitric oxide from macrophages increased tumor cell sensitivity to DOX.

### Discussion

MHySN, made using a highly-controllable sol-gel process, possess the ability to host, protect and controllably deliver diverse types of cargoes due to their fine-tunable structure, porosity and surface chemistry. The ability to tune pore size and volume, as well as the surface area, can be tailored for various cargo with diverse properties (e.g., small drugs, medium-sized enzymes, and large complex-proteins). The hybridization of inorganic silica with organosilanes confers activity to the particle of choice with copious options for modification. For instance, inserting light-, pH- and/or redox-responsive organic functions that exhibit triggered release of payloads by controllable charge change/reversal, or successive degradation, is made possible. Also, incorporating aromatics, aliphatics and fluorinated moieties tunes the hydrophobic-hydrophilic balance of the pore of the MHySNs, thus allowing tunable loading capacity and/or release kinetics. MHySN have also overcome cargo capacity and diversity limitations as they are able to adsorb individual or multiple cargos (imaging agents, peptides, siRNAs, and drugs with different physicochemical properties) into their mesoporous siliceous cores that are protected and retained by the supported lipid bilayer. Compared to FDA-approved liposomal doxorubicin, in vitro studies have demonstrated that lipid-coated MSN can deliver 1,000-fold more doxorubicin per particleA Furthermore, cargos are retained until efficiently delivered to target cell cytosolic intracellular compartments by ionic alterations or pH-triggered destabilization of the lipid bilayer, and endosomal swelling and disruption orchestrated by endosomolytic peptides incorporated in the lipid bilayer.

In this work, MHySN were used to co-load DOX (cisplatin or oxaliplatin) and CpG ODN within large-pore (e.g., 8-15 nm) MHySN. Different strategies were used to achieve successful co-loading of cargoes of different sizes and charges, such as loading conditions and incorporating specific functions (e.g., organic components, for example, hydrazone linkers) in the MHySN for better retention, higher loading extents, and/or pH-dependent release. Additionally, the loaded MHySN can be encapsulated within a preformed liposomal system containing the adjuvant, e.g., MPL, that assists with loading higher amounts of cargo, creates a seal for the pores to prevent premature leakage, confers to the whole system an enhanced stability in bio-relevant environments, and incorporates environmentally-triggered release kinetics.

### EXAMPLE 2

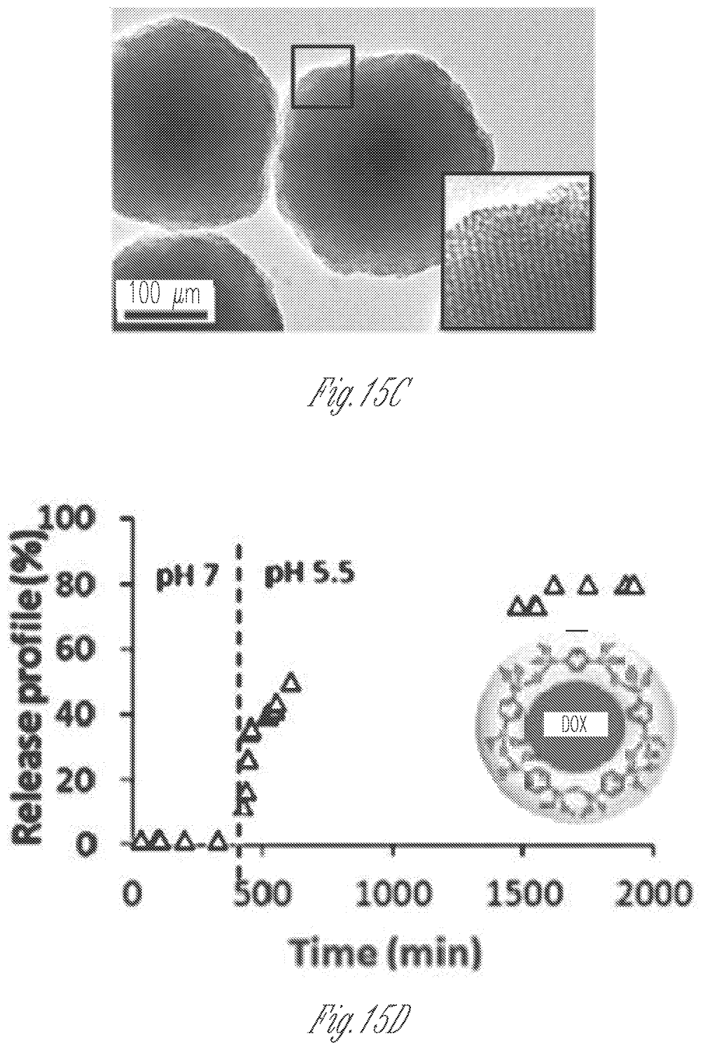

Defining molecular and imaging profiles to guide patient therapy. This project seeks to increase the understanding of biological responses to therapy and to promote clinically noninvasive imaging of these events. Functional noninvasive imaging profiles and biological markers guide evaluation of patient responses to therapy leading to early termination of treatment regimens prior to adverse events or more aggressive treatment regimens for positive responders. Further, increased fundamental understanding of the processes underlying the use of nanotechnology and checkpoint blockade will support the development of robust, safe immunotherapy approaches that help us in our fight against cancer. Structural and functional data obtained with noninvasive imaging includes tissue information, such as cell density, necrosis, metabolic activity, vascular permeability and tissue perfusion. Preliminary positron emission tomography (PET) imaging studies show high ¹⁸F-fluorodeoxyglucose (¹⁸F-FDG) uptake (metabolic activity) in cancer cells with location mimicking cancer cell density (IVIS) and spatial location of large, perfusable blood vessels [dynamic contrast-enhanced magnetic resonance imaging (DCE-MRI)] in mice with 4T1 tumors FIG. 13A). Computed tomography (CT) and intravital imaging of 4T1 tumors show large, perfusable blood vessels restricted to the tumor periphery (FIG. 13B), supporting vascular accessibility to locations abundant in tumor cells.

Logic-embedded immunogenic nanoparticle platform. Advantages of the MHySN platform include the well-known assets of liposome formulations (low inherent toxicity, tailored environmentally responsive lipid bilayers) and the high stability and capacity for loading and simultaneous delivery of multiple cargos by porous nanomaterials, Logic-embedded MHySN sequentially and logically presents cargo, beginning with MPL for engagement of TLR-4 on immune cells. Once internalized, the dissociation of the supported lipid bilayer in the acidic endosome exposes or releases TLR-9 ligand (e.g., CpG ODN) that in turn engages receptors within the endolysosomal compartment. Transfer of DOX from immune cells to tumor cells has been proposed to occur by release from dead cells. Choi et al. demonstrated that systemically-injected peritoneal macrophages loaded with liposomal DOX successfully migrate to tumors and inhibit tumor growth. Furthermore, Soma et al. showed that DOX-induced secretion of nitric oxide from macrophages increased tumor cell sensitivity to DOX.

The surface-rendered 30 confocal image in FIG. 14A shows a cell internalized MHySN consisting of a supported lipid bilayer and mesoporous silica core co-loaded with DOX (green) and Cy5-nucleic acid (red). FIG. 14B is a graph of CD40 expression on the surface of RAW macrophages 24 h after introduction of MHySN to the culture. Here CD40 was used as a metric for the functional presentation of MPL in the supported lipid bilayer. MHySN created using liposome sonication and unmodified mesoporous silica cores were superior to MHySN created using extrusion and carboxy-modified mesoporous silica. Alternative mesoporous organosilane cores have a high affinity for DOX with benzene-based silsesquioxane nanoparticles exhibiting a 75% loading efficiency. Release of DOX is highly pH-sensitive negating the need for pH-responsive linkers (FIG. 14C). We recently demonstrated high CRISPR Cas9 editing using a supported lipid bilayer mesoporous silsesquioxane nanoparticles to deliver the ribonucleoprotein to reporter lung cancer cells. Fully loaded MHySN are roughly 175-275 nm with polydispersities below 0.18

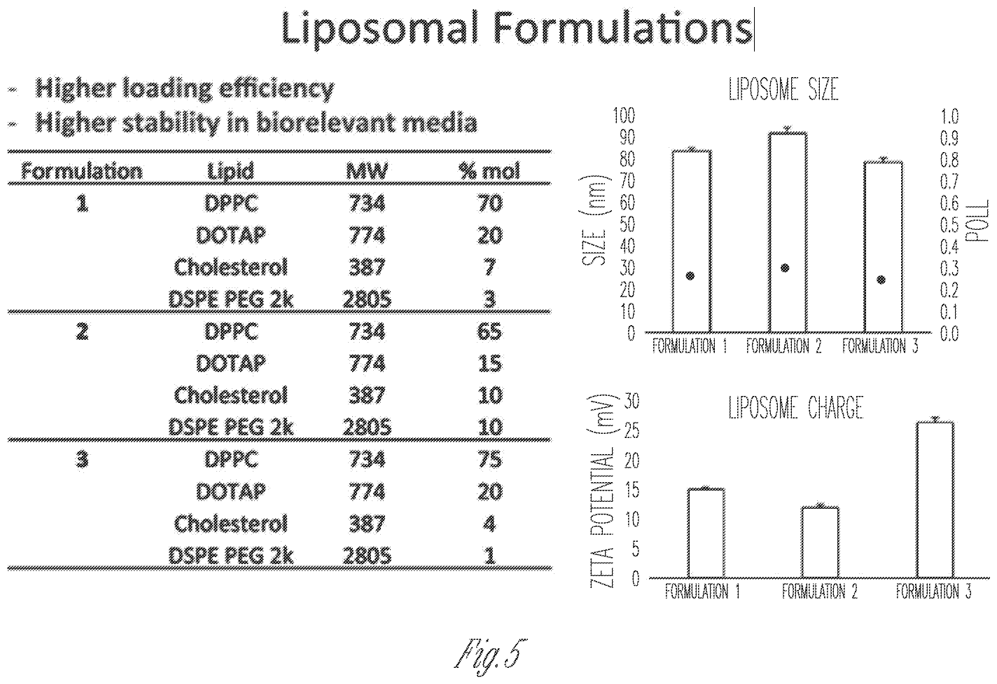

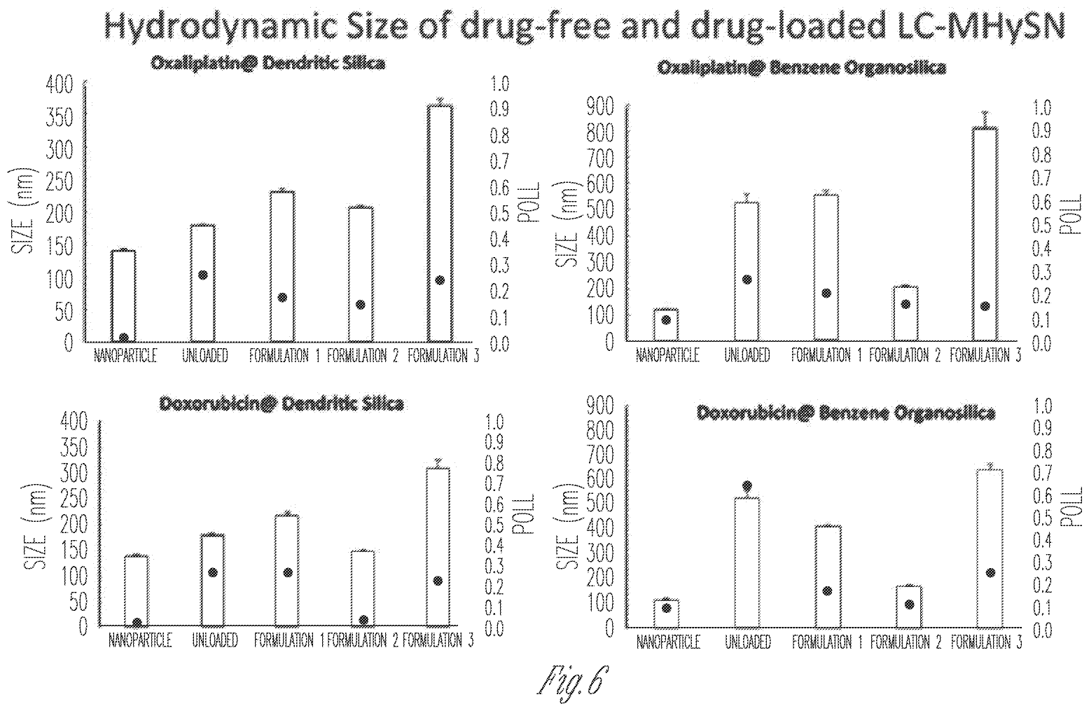

Intercellular transport of NPs and chemotherapeutics. The release of DOX from macrophages could be triggered by cell death or may alternatively involve intercellular transport of DOX via secretion of DOX containing membrane-bound vesicles (biovesicles) or direct cell-to-cell transfer. Cytotoxic NPs are secreted from donor endothelial cells in biovesicles that are subsequently internalized by nave acceptor cells. Furthermore, here we present unpublished data showing the intercellular transfer of mesoporous silica nanoparticles (MSN) and DOX, both within the macrophage population (homotypic; FIG. 15A) and between macrophages and cancer cells (heterotypic; FIG. 15B) via cellular cytoplasmic bridges known as tunneling nanotubes (TNT). RAW macrophages pre-incubated with NPs are capable of transporting NPs to nave macrophages or to cancer cells (e.g., human HeLa cervical cancer cells). In addition, active transfer of DOX between macrophages occurs through INTs, as shown in FIG. 4c, with the white arrow highlighting the DOX (red)-filled TNT. Within the tumor, recipient cancer cells have greater susceptibility to DOX than macrophages based on high rates of metabolic activity and inhibition of macromolecular biosynthesis.

### EXAMPLE 3

MHySN, made using a highly-controllable sol-gel process, possess the ability to host, protect and controllably deliver diverse types of cargoes due to their fine-tunable structure, porosity and surface chemistry. The ability to tune pore size and volume, as well as the surface area, can be tailored for various cargo with diverse properties (e.g.

small drugs, medium-sized enzymes, and large complex-proteins). The hybridization of inorganic silica with organosilanes confers activity to the particle of choice with copious options for modification. For instance, inserting light-, pH- and/or redox-responsive organic functions that exhibit triggered release of payloads by controllable charge change/reversal, or successive degradation, is made possible. Also, incorporating aromatics, aliphatics and fluorinated moieties tunes the hydrophobic-hydrophilic balance of the pore of the MHySNs, thus allowing tunable loading capacity and/or release kinetics. MHySN have also overcome cargo capacity and diversity limitations as they are able to adsorb individual or multiple cargos (imaging agents, peptides, siRNAs, and drugs with different physicochemical properties) into their mesoporous siliceous cores that are protected and retained by the supported lipid bilayer. Compared to FDA-approved liposomal doxorubicin, in vitro studies have demonstrated that lipid-coated MSN can deliver 1,000-fold more doxorubicin per particie.⁴⁰ Furthermore, cargos are retained until efficiently delivered to target cell cytosolic intracellular compartments by ionic alterations or pH-triggered destabilization of the lipid bilayer, and endosomal swelling and disruption orchestrated by endosomolytic peptides incorporated in the lipid bilayer.

MHySN are used to co-load DOX and CpG ODN within large-pore (8-15 nm) MHySN. Co-loading of cargoes of different sizes and charges in MHySNs, such as loading conditions and incorporating specific functions (e.g., organic components, hydrazone linkers) in the MHySN for better retention, higher loading extents, and pH-dependent release. Additionally, the loaded MHySN may be encapsulated within a preformed liposomal system containing the adjuvant MPL that assists with loading higher amounts of cargo, creates a seal for the pores to prevent premature leakage, confers to the whole system an enhanced stability in bio-relevant environments, and incorporates environmentally-triggered release kinetics.

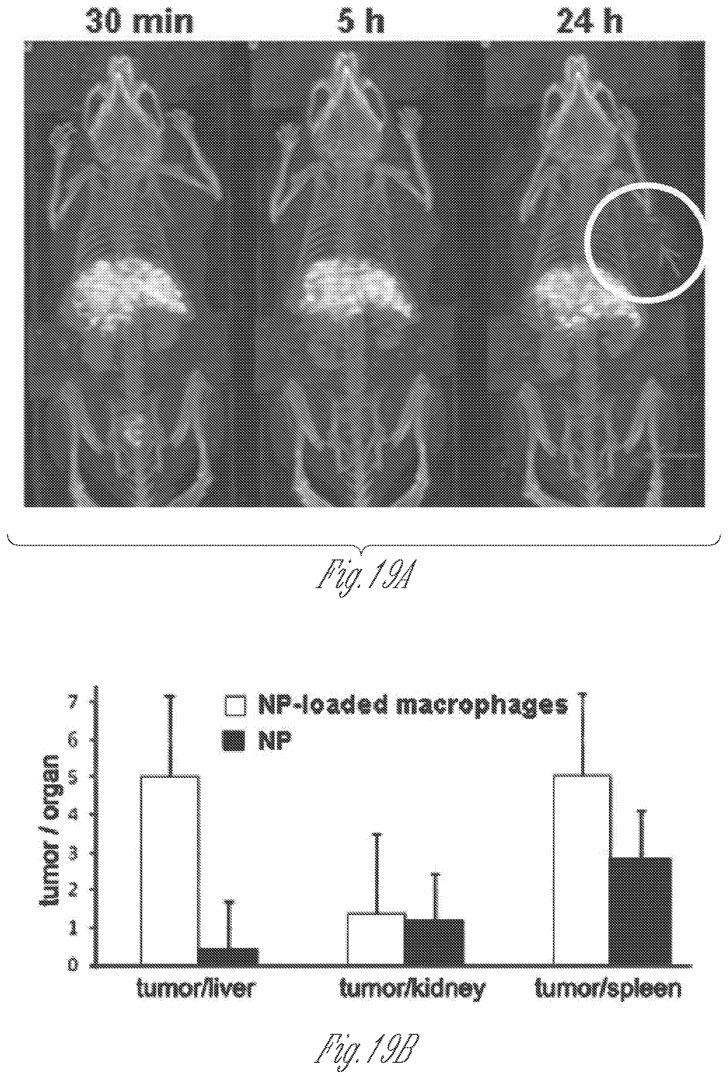

MHySN formulation for DOX loading and release, and CpG loading into the mesoporous core, and fusion of the MPL containing supported lipid bilayer. Design and fabrication of MHySN. The system includes a mesoporous siliceous core for dual loading of hydrophilic DOX hydrochloride and hydrophilic CpG ODN encapsulated within an MPL-containing zwitterionic lipid bilayer (FIG. 18).

a. Constructing hybrid silica cores. The pore size will be adapted to accommodate the molecular-nucleic acid cargo. A high control of the core structure and its characteristics can be tuned to the benefit of loading. In one embodiment, the particles will have a core size of around 100 nm to preserve an acceptable range for bioapplications after addition of all the components (generally ca. 120 nm). Surface charge and core chemistry are integral to the assembly of monodisperse MHySN. One or multiple adapted organic bridges may be incorporated within the particle pore walls (e.g., benzene or ethane) to load the DOX on one hand, and the CpG on the other hand, but also to favor the fusion of the lipid bilayer. The loading process is mainly governed by electrostatic interactions but also by the complex hydrophilic/hydrophobic character of molecules with the surface of interest involving dipole-dipole van der Waals interactions and potential H-bonding. The excellent functional fertility of MHySN is an important factor n to tune the charge and other interactions in favor of the loading.

b. Cargo loading. Considerations include a dual loading-friendly environment, avoiding saturation of the pores by only one molecule, and loading the correct ratio of molecules to nucleic acids. Also, the solvent, pH, and concentration of loading solution, time, and temperature are selected for each application. Different molecules may be loaded together or sequentially. Short incubation times for co-loading may be used for premixed molecules such as protein and DNA.

c. Selecting lipid-supported bilayer. The selected lipid composition is: stable when fused to the loaded MHySN; and ii) prevent premature leakage of the payload. The best ratio of lipid to loaded MHySN is used.

Physico-chemical characterization. For lead candidates, in vitro techniques established by the NCI Nanotechnology Characterization Laboratory (NCL) including dynamic light scattering, cryo-TEM, and zeta-potential measurements will be conducted to assess colloidal stability. The sorption theories of Brunauer-Emmet-Teller (BET) and

Barrett-Joyner-Halenda (BJH) are used to respectively calculate the surface area and the average pore size of the particles based on the nitrogen sorption isotherm.

Functional evaluation of MHySN formulations. The murine RAW macrophage cell line or bone marrow derived DC are used to study TLR-mediated activation of APC. MHySN are incubated with cells and viability, upregulation of costimulatory molecules, and DOX release will be examined using fluorescent microscopy and flow cytometry at 1, 6, 24 and 48 hours.

### EXAMPLE 4

Macrophages are labeled with¹¹¹In for in vivo quantitation using SPECT imaging and gamma counting of organs. Co-localization of adoptively transferred HSV1-tk transformed macrophages with MHySN will be evaluated using ¹⁸F-FIAU and PET imaging.

Preliminary data. Preliminary studies support the ability to label MHySN with indium-111 or DyLight 650 for monitoring biodistribution in mice using SPECT or fluorescence imaging, respectively. Splenic macrophages were co-incubated with ¹¹¹In and DyLight 650 labeled NPs (DOPC-DOPS-silica), followed by adoptive transfer of cells into PyMT mice for successful quantitation in organs and imaging of biodistribution (FIG. 14). In a separate experiment, tumor accumulation of DyLight 650-DOPC-DOTAP-silica NPs was evaluated in PyMT mice administered intravenous NP-loaded macrophages or free NPs. Post-injection (24 hours), organs were excised, digested, and the tissue was analyzed by flow cytometry. Accumulation of macrophage-carried NPs in the tumor, relative to liver and spleen, was greater than NPs administered by direct intravenous injection (FIG. 13B).

Evaluate ¹¹¹In-MHySN or ¹⁴C-MHySN pharmacokinetics and biodistribution using gamma counting and whole-body SPECT/CT imaging Radiolabeling of MHySN. To increase sensitivity, preformed liposomal NPs are labeled with ¹¹¹In using a lipid-soluble metal complex of the gamma emitter. Metal chelation of ¹¹¹In to tropolone will be achieved by mixing ¹¹¹In-chloride and tropolone for 15 minutes at pH 7.0-7.5. ¹¹¹In-tropolone solution will slowly be added to liposomes at room temperature. ¹¹¹In labeling efficiency and stability will be studied prior to use in animals. Alternatively, covalent anchoring of radiolabels on the nanosystem will be performed using a free radical polymerization reaction of ¹⁴C-acrylic acid on allyl-bearing MHySN. This high-fidelity method hinders any leaching of radioactive materials and prevent false signaling. This option is possible thanks to the versatility and effective functionalization of the mesoporous core in the proposed system.

Pharmacokinetic and biodistribution studies. Orthotopic 4T1 tumors are created using the parental cell line (hereafter 4T1 null) in female 8-10 week-old BALB/c mice by injection with 1×10⁵ cells in PBS into the fourth inguinal mammary gland using IACUC approved protocols. Based on the majority of breast cancer patients being female, studies with tumors will use female mice. Mice arte provided food and water ad labium, To estimate the number of mice needed to accomplish the proposed work, data obtained from preliminary studies using adjuvant liposomes was used to estimate the proposed effect size, standard deviations and correlation coefficients, and computed minimum sample sizes using a power set at 90%, type 1 error set at 5% and based on a two-sided T-test. Based on signal to noise ratios of 1.8 and 1.5, 6-10 mice are needed per group for therapeutic efficacy and biodistribution studies. Above the indicated number of mice, we are requesting 10% additional mice to account for unexpected deaths, tumor implantation failure (typically less than 5%) and for splenocyte harvest. When tumors are approximately 500-750 mm³, PBS dispersions of ¹¹¹In-(or ¹⁴C)-MHySN, adoptively transferred HSV1-tk transduced macrophages with internalized ¹¹¹In-MHySN, or PEGylated ¹¹¹In-MHySN (at an approximate dose of 500 μCi ¹¹¹In) are administered by intravenous injection with n=6 mice/group. At 15 and 30 min, 1, 3, 5, 24, and 48 hours mice are sacrificed and plasma and organs (tumor, liver, spleen, lymph nodes, lungs, heart brain, kidney) counted using a gamma scintillation counter (3 groups×6 mice/group×7 time points=126 female mice). Blood concentration profiles, half-life, elimination rate, area under the curve (AUC) and mean retention time (MRT) of MHySN in tumor tissue are evaluated.

Whole body distribution will be imaged by SPECT (co-registered to CT) at 1 and 24 hours post-injection of ¹¹¹In-MHySN using a NanoSPECT/CT® Small Animal In Vivo Imager (Bioscan, Inc, Washington, D.C.) in cohorts of 5 female mice per group as above (Total=15 mice). CT acquisition is achieved using 180 projections with a pitch of 1.5 and Helical SPECT acquisition will include 32 projections with varying time per projection. Helical SPECT/CT images are reconstructed into a series of 2D axial images. Accumulation of MHYSN in SPECT images are determined by analyzing volumes of interest (VOIs) and determining tissue loads (%injected dose per gram) for organs (liver, spleen, kidneys, lungs) and tissues (tumor, lymph nodes, muscle) using VivoQuant 2.00 software (inviCRO, LLC).

Evaluate co-localization of adoptively-transferred macrophages and MHySN cargo in mice. Carrier macrophages (splenic or RAW 264.7 cells) are either transiently transduced with the herpes simplex virus type 1 thymidine kinase (HSV1-tk) pLOX-gfp-iresTK plasmid (addgene), or stably transduced with TK-RFP Lentiviral particles (GenTarget Inc.). RAW 264.7 cells were used because they are macrophage cells originated from the ascites of a BALB/c mouse with a leukemia virus-induced tumor. Alternatively, newly isolated splenic macrophages are transfected with the pLOX-gfp-iresTK plasmid. Splenic macrophages are superior to bone marrow-derived macrophages with respect to tumor infiltration. Prior to animal use, transfection/transduction of cells will be optimized and validated using flow cytometry (RFP or GFP detection). Prier to animal imaging, uptake of the radiotracer in transduced and non-transduced parental RAW cells and primary splenic macrophages will be compared. Brust et al. (2001) reported a 28-fold increase in reporter expression 2 h after ¹²⁴I-2′-fluoro-2′-deoxy-5-iodo-1-beta-D-arabinofurariosyluracil (¹²⁴I-FIAU)injection in glioblastoma cells. They reported that PET imaging of in vivo uptake of the two tracers (¹⁸F-FHPG and ¹²⁴I-FIAU) in mice with transduced 4T1 tumors showed higher accumulation of radioactivity using ¹²⁴I-FIAU compared to ¹⁸F-FHPG. Based on the higher uptake of the uracil nucleoside FIAU, ¹²⁴I-FIAU was used as the reporter probe for PET imaaing of HSV1-tk gene expression in adoptively transferred macrophages. If transient transfections are used,¹¹¹In-MHySN loaded with plasmid will be presented to cells for 3 hours, washed to remove non-internalized NPs, and injected intravenously above. Post SPECT imaging (as described previously), mice will be administered an intravenous injection of ¹²⁴I-FIAU radiotracer. FIAU is a substrate for the TK gene and expression will be monitored 2 hours post administration using PET/CT imaging. Quantitative imaging of cells expressing TK has been demonstrated.⁴⁵ Co-registration of PET, SPECT, and CT images enables the degree of colocalization of adoptively transferred macrophages and MHySN.

### EXAMPLE 5

In vivo acute toxicity over a range of doses is determined and repeat dose toxicity studies are performed to evaluate the maximum tolerated dose (MTD) of DOX-loaded and control MHySN in immune competent BALB/c mice to guide dosing in efficacy studies and such that Phase I/II studies can be designed. Based on the high loading capacity and pH-sensitive release of DOX in MHySN, the MID of MHySN will likely exceed that reported for Doxil®, and the MTD of unloaded MHySN will likely exceed that of DOX-loaded MHySN.

Silicon dioxide NPs comprise 8% of all air born NPs in ambient air, making exposure to humans nonavoidable. Although MHySN are not presented as an aerosol, the abundance of Si in the environment increases the necessity for studying single and repeat dose effects of silicious NPs in the control unloaded and drug-loaded states. Toxicity studies are performed in male and female mice so that the data can be applied to other cancer models in the future.

In vitro toxicity studies. Cell type differences in susceptibility to control and DOX-loaded NPs are evaluated using stromal (endothelial and fibroblast), myeloid (M1 and M2 macrophages), and cancer cells. Cell viability (alamar blue), cell membrane integrity (propidium iodide or DRAQ 7), apoptosis (annexin V), and generation of reactive oxygen species (ROS; 2′,7′-dichlorofluorescein diacetate) are evaluated using a plate reader and flow cytometry across MHySN doses ranging from 1 to 100 pg/rtil at 1, 3, 6, 24, 48 and 72 hours. Inflammatory biomarkers (IL-1β, IL-6, TNF-α) will also be evaluated in cell supernatant at 24 hours post introduction of MHySNs. The IC50 for DOX is 0.08 μg/ml, while that of dendrimer-hydrazone DOX is 1.4 μg/ml. IC50 values for MHySN DOX will be derived from study results.

In vivo toxicology studies. The non-GLP toxicology studies have a dual purpose. First, the maximum tolerated dose (MTD) of an exemplary MHySN formulation (with and without DOX compared to no treatment controls) is determined when administered to 8-10 week old immunocompetent BALE/c male and female mice as a single intravenous injection. Mice (6 per group) are given a single injection of the lead MHySN over a wide range of doses. The MTD for DOX is 6 mg/kg with liposomal or dendrimer (via hydrazine linkers) delivered DOX being 10× less toxic with an effective single therapeutic dose of 20 mg/kg. Low, mid and high doses of DOX-MHySN are within the range 5-100 mg DOX/kg. Mice are monitored for treatment-related complications, including body weight, activity level, hematological parameters, organ weights and histopathological findings. Blood is collected for clinical pathology (hematology and clinical chemistry parameters) and anatomic pathology (necropsy, gross observations, and histopathology) evaluations.

Once the single dose MTD is identified, a repeat dose study is performed. The purpose of the repeat dose study is to identify possible target organs for both the drug-loaded and unloaded control MHySN. Information obtained from this study guides dose selection for efficacy studies. Four groups of mice are injected intravenous with the lead MHySN formulation at the MTD, as determined in the single dose dose-ranging study, and fractional doses thereof on an appropriate dosing schedule. Clinical observations are made throughout the study. All animals are euthanized following the observation period. Parameters to observe during dosing include body weight, activity and fur ruffling. At the end of the study, organs are collected, weighed and histopathology evaluation is performed on the spleen, liver, and lymph nodes with an emphasis on the presence of vacuoles, necrosis, immune cell infiltration. Sections of the spleen are used to evaluate B and T cell populations (numberig tissue and phenotype to include helper, cytolytic and natural killer cells). Clinical chemistry analysis for liver damage includes alkaline phosphatase, alanine transaminase, and aspartate transaminase.

### EXAMPLE 6

Evaluate the therapeutic efficacy and immunogenicity of MHySN and anti-PD-1 antibody using variants of the orthotopic syngeneic 4T1 and transgenic MMTV-PyMT mouse models of breast adenocarcinoma. Monitor physiological responses to therapy through noninvasive imaging with an emphasis on tumor growth, tumor perfusion and metabolic rates using IVIS and calipers, MRI and PET imaging. Define associated changes in tumor tissue architecture, immunocytes, cytokines, markers of activation, and tissue necrosis using electron and fluorescent microscopy, flow cytometry, cytokine (Luminex) and antibody (ELISA) analysis, MHySN TLR ligand-mediated activation of immunocytes and delivery of pH-triggered release of immunogenic DOX combined with immune checkpoint blockade ablates tumor cells and stimulate antitumor immunity superior to single-agent MHySN therapy.

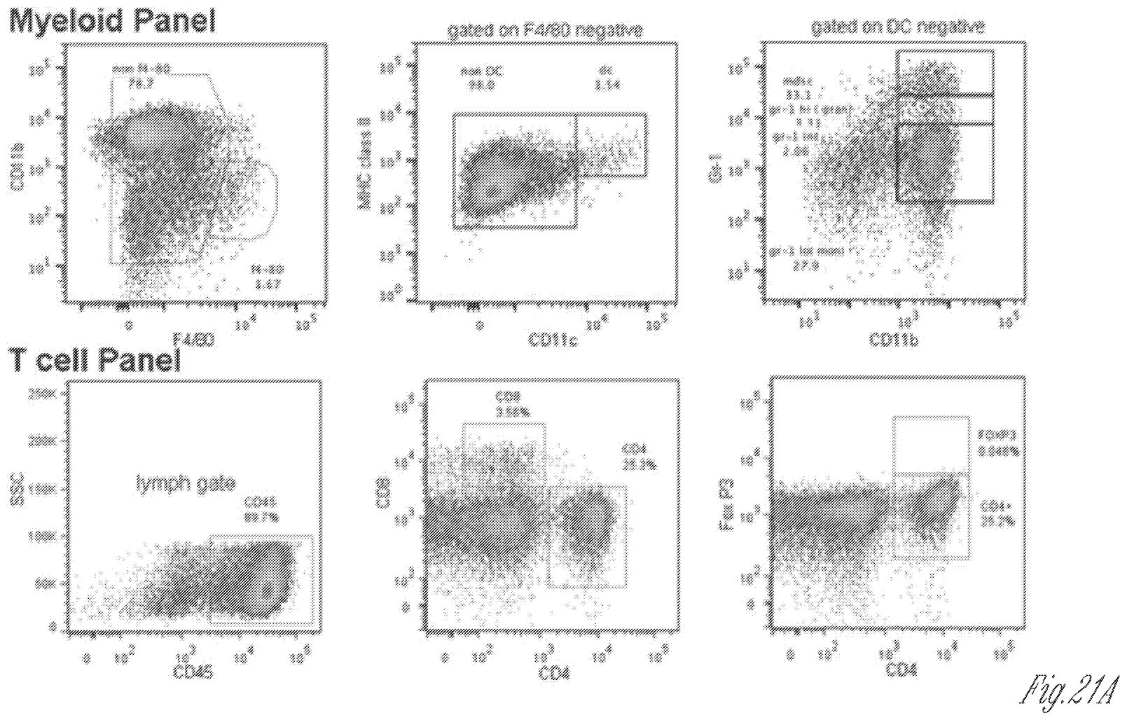

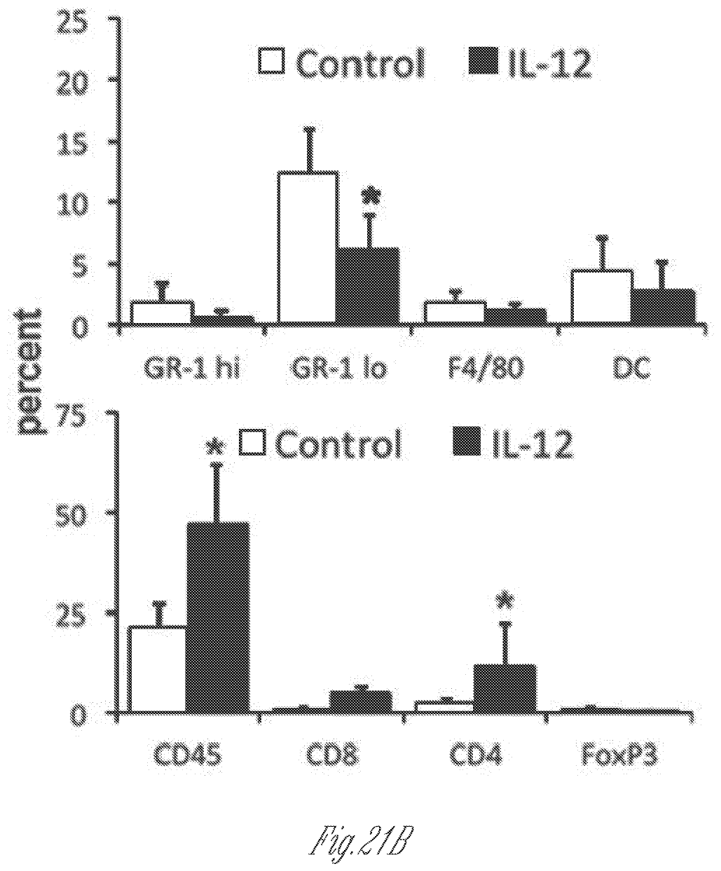

Preliminary data. Intratumoral delivery of cationic liposomes presenting MPL and IL-12 blocks 4T1 tumor growth, cell proliferation, and alters the cytokine milieu of the tumor microenvironment (FIG. 20). Th-1 cytokines in turn impact myeloid phenotype, favoring M1 macrophages. While MPL therapy elevated levels of inducible nitric oxide synthase (iNOS) 3-fold above basal levels in the tumor, combination MPL and IL-12 cationic liposome therapy stimulated a 7-fold increase, supporting the observed cell cycle arrest (loss of Ki-67 expression) and apoptosis (TUNEL positive) and arguing in favor of our currently proposed combination TLR ligand therapy to activate dual signaling pathways. In mice bearing multiple tumors, the growth of distal, untreated tumors mirrored that of liposome-treated tumors, supporting the presence of a systemic immune response.

Mouse models. Female MMTV-PyMT mice spontaneously develop mammary epithelial tumors in FVB mice that mimic tumor progression in human breast cancer, with tumors developing in mice over the course of 10-12 weeks. Based on lack of, or delayed, tumor growth in male MMTV-PyMT mice this study will use female mice. To explore altered biological responses to parental and virally-transduced 4T1 models to immune therapy, tumors are established using either 4T1 parental or 4T1 luc cancer cells and tdTomato Red/luc cells in syngeneic female BALB/c mice as described above.

Immune checkpoint blockade. Anti-PD-1 antibody is purchased from BioXCell (clone J43) or PD-1 is purified from the hybridoma clone C1-G4, a gift from Dr. Lieping Chen at Yale University. Briefly, hybridoma supernatant is ammonium sulfate precipitated to 45% overnight at 4° C. and dialyzed against PBS for 24 hours. Serial dilutions of each antibody are tested for binding to Jurkat cells or splenocytes and for functional blockade of T cell activity. CD4⁺ and CD8⁺ T cells from murine splenocytes will be purified using Miltenyi MACS beads followed by activation with plate-bound anti-CD3 antibody (3 μg/ml for 4 days). For therapeutic efficacy studies, mice are administered PBS control or 200 pg anti-PD-1 or control anti-hamster IgG antibody in 100 μl PBS intraperitoneally (i.p.; shown to elicit high CD8+ T cells in the tumor) once a week, starting 10 days after tumor initiation in mice with 4T1 tumors or when tumors become palpable in MMTV-PyMT in mice.

Treatment regimen. Ten days post-injection of 4T1 or 4T1 tdlomato red luc breast cancer cells or when 4T1 and MMTV-PyMT tumors become palpable, mice begin weekly treatment with isotype control or anti-PD-1 antibody (i.p.) and MHySN loaded with DOX, MPL and CpG (i.v.). Vehicle controls, single-agent MHySN, and fully loaded MHySN are included, the latter with and without anti-PD-1 antibody. Two BALB/c 4T1 tumor models are used to test 9 treatment groups with 10 mice per group; requiring a total of 180 female 8 week old BALB/c mice. In addition, the NP formulation is compared to standard of care agents DOXIL®® and DOX. All NP and DOX agents will be delivered by intravenous administration of 100 μl in PBS. In addition, the fully-loaded MHySN and anti-PD-1 antibody (combined and as single-agents) are tested in the genetically-distinct MMTV-PyMT mouse model requiring 20 female MMTV-PyMT mice. As shown in FIG. 20, tumor growth is monitored by caliper measurements and based on luciferase expression using the Xenogen IVIS System to detect bioluminescence following i.p. injection of 150 mg/kg RediJect D-Luciferin. Body weight and body score will be recorded 3×/week. Mice will be euthanized upon signs of morbidity (body score≤2) or when tumors are greater than 20 mm diameter. Blood will be collected by cardiac puncture, and tumor, draining lymph nodes, and spleen are collected for weight and size measurements, with immediate freezing for fluorescent IHC analysis of cellular phenotypes, or used for tissue dissociation for cellular phenotyping by flow cytometry or cytokine analysis. BALB/c mice with no tumor burden are monitored over time for tumor recurrence and select animals will be re-challenged at 3 months by injection of 4T1 cells into the contralateral mammary fat pad. The metastatic burden is evaluated in lungs and liver of all mice by excising the tissue and staining with Coomassie blue dye (tumor nodules don't stain). Surface nodules and nodules present within tissue serial sections are obtained every 5 mm using a microtome and counted. Alternatively, foci are counted in 5 randomly selected sections per specimen following H&E staining.

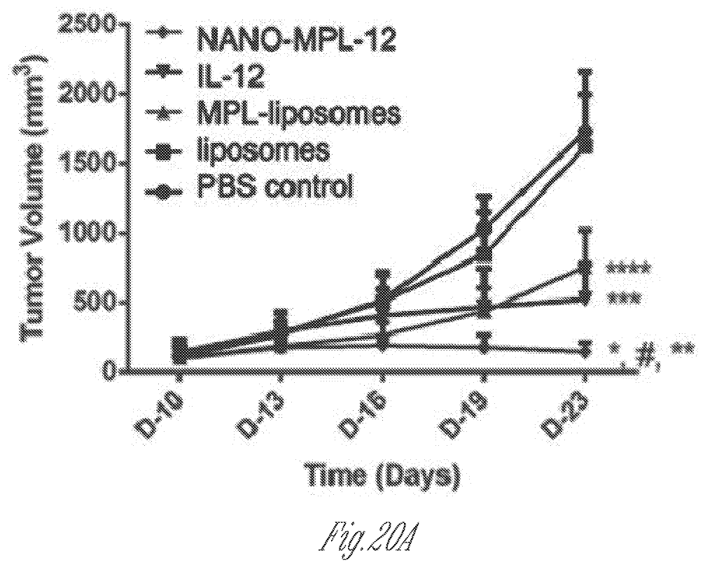

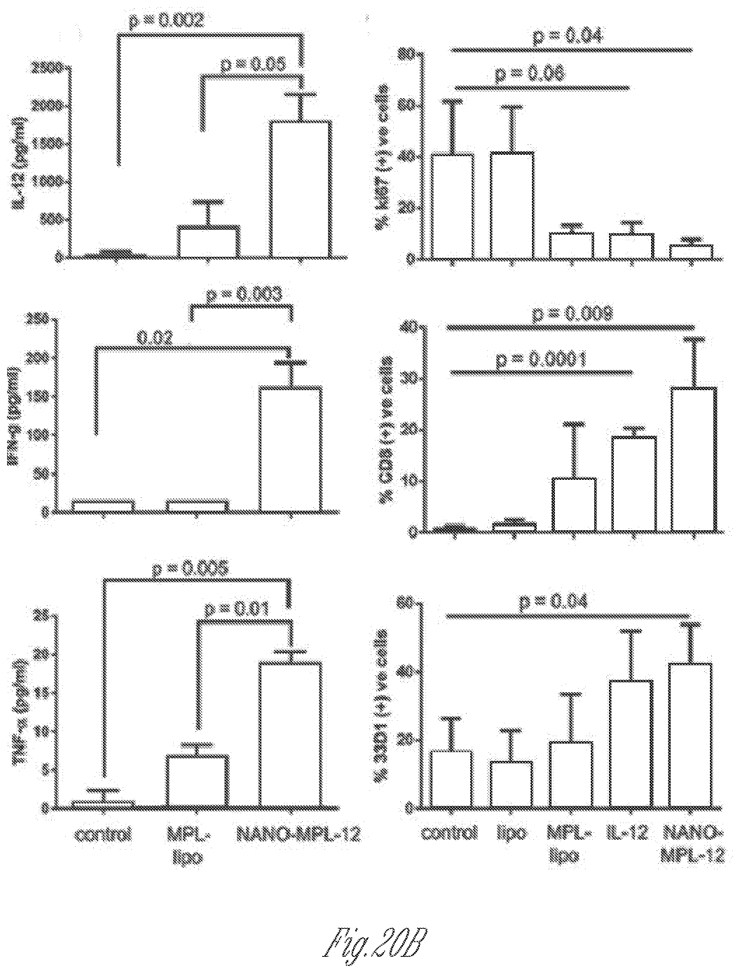

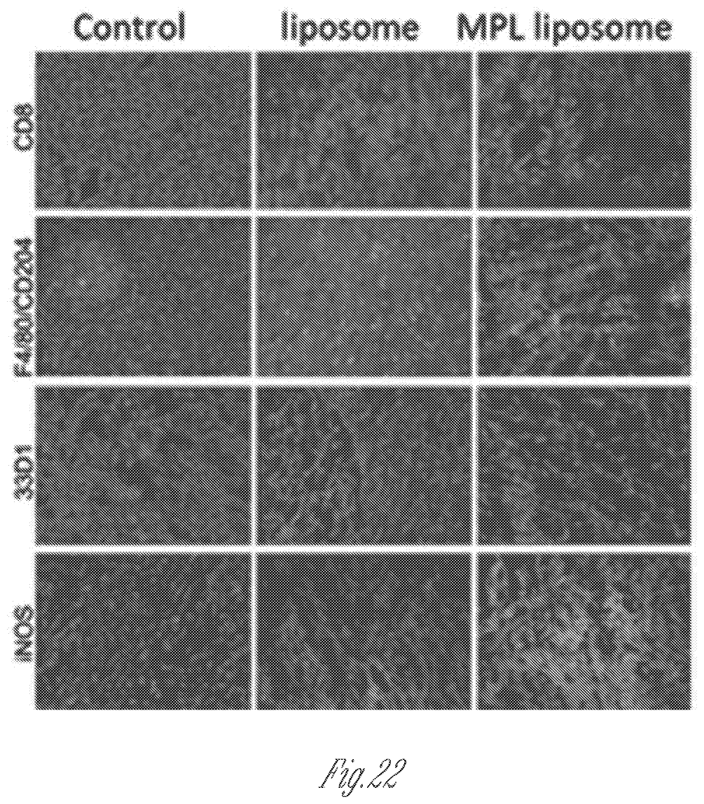

Tumor phenotyping. Flow cytometry and immunohistochemistry are used to characterize PD-L1 expression and the presence of effector and regulatory T cells, myeloid and DC, vascularity and stromal cells in tumors and lymphatic tissue. Immunosuppressive regulators in the tumor include T regulatory (Treg) and myeloid-derived tumor cells (MDSC). Tregs (CD4⁺/FoxP3⁺) and MDSC [CD11b⁺/Gr-1(Ly6-CIG)⁺] suppress effective antitumor immune responses. CD4⁺ and CD8⁺ T cells are the primary adaptive immune cell mediators within the tumor and the proportion of T cell subsets and DC present in the tumor plays a critical role in tumor rejection. Preliminary studies show that IL-12 decreases MDSC and increases CD4⁺ and CD8⁺ T cells in the tumor 24 h after treatment (FIG. 22). Single cell suspensions are prepared from tumors and spleen. After Fc receptor blockade with anti-CD16/CD32 antibodies in 1% mouse serum/1 mM EDTA, cells are permeabilized for intracellular staining or stained directly with antibodies identifying discrete immune cell populations, as described below. Immunocyte subsets will be identified with combinations of surface markers: effector and regulatory T cells [CD8⁺, CD4^{+; FoxP}3⁺ (Treg)], macrophages (Gr1^{+, CD}204, F4/80), myeloid and granulocyte derived suppressor cells (Gr1⁺CD11b⁺ or Ly6C/Ly6G/CD11b⁺) and dendritic cells (33D1, CD103, CD11c⁺), and natural killer (NK) cells (NK1.1⁺CD3⁻). Specificity of binding to naïve or memory/effector cells is evaluated using fluorescent antibodies that recognize CD45RA, CCR7, and FOXP3. Tissues will be quickly frozen in O.C.T. and sections fixed and fluorescent-labeled with primary antibodies to include those specific for the above mentioned immune cell populations as well as smooth muscle actin, E-cadherin, and PECAM (CD31), specific for fibroblasts, cancer cells, and blood vessels. Apoptotic tumor cells are detected using DeadEnd Colorimetric TUNEL System, and proliferating cells will be identified using Ki-67. Tissues will be mounted in ProLong Gold containing DAPI, and images are acquired with a fluorescent microscope at 20× or 60× magnification. Fluorescent micrographs of immunocyte infiltration into 4T1 tumors following treatment with cationic MPL liposomes are shown in FIG. 19.

Profile markers of phenotypic polarization and functional activation including macrophages (markers of M1 vs. pro-tumor M2 phenotype). MDSC (functional markers), CD4⁺ helper T cells (Th1 vs Th2), and CTL (cytolytic activation markers. In addition to the specific identification of immunocyte populations, additional understanding of the intratumoral immune milieu can be obtained by examining markers of functional activation and phenotypic polarization. The following activation markers will be used for macrophages: M1-iNOS, TNF-α; and M2arginase and IL-4; MDSC (functional markers PD-L1, iNOS, and arginase), CD4⁺ helper T cells (Th1 vs Th2 intracellular cytokine staining), and CTL (cytolytic markers perforin and granzyme B). As critical mediators of tumor-induced immunosuppression, negative regulatory molecule expression on CD4⁺ and CD8⁺ T cells, including PD-1, CTLA4, and LAG-3, is profiled. Single cell suspensions prepared from lymphatic organs [spleen and LNs (axillary and inguinal)] are used to measure T and B cell activation. Cells will be stimulated for 5 days at 37° C. with mitomycin-treated tumor cells at a 10:1 ratio in the presence of 10U/ml recombinant mouse IL-2. Cells are analyzed by flow cytometry following labeling with anti-mouse CD4, CD8a (T cells) and CD45R (B220: B cells).

Analyze serum cytokine and antibody levels in response to therapy. Serum analysis of cytokine and angiogenic factors is 24-48 h after the second weekly NP treatment. In addition, for tissue cytokine analysis, tumor and spleen are pulverized and powders dissolved in cold PBS and centrifuged. Protein samples are adjusted to 100 μg/mland cytokines detected with the LUMINEX® 100/200 system. Markers analyzed include chemokines (5-Plex Panel) and custom cytokine panels that include VEGF, Th1 and Th2 analysis. Humoral antibody responses are evaluated in serum using goat anti-mouse IgG1 or IgG2a, followed by HRP-conjugated anti-goat IgG,

MRI-PET imaging to characterize cellularity, tissue permeability and metabolic activity. Recent technological advances have led to the rapid development and implementation of PET/MR imaging. MRI and PET are combined to comprehensively evaluate tumor response to therapy. In addition to the presence of immunocytes, early inflammatory events are associated with an increase in local vessel permeability due to the release of cytokines, chemokines, and leukotrienes by resident inflammatory and endothelial cells. DCE-MRI is used to derive data on tissue perfusion, microvascular vessel wall permeability, and tissue cellularity, markers of tissue integrity and indicators of treatment response. ¹⁸F-FDG (2-deoxy-2-¹⁸F-fluoro-D-glucose) is the most commonly used PET imaging tracer and has been used for tumor detection and staging based on high glucose metabolism in malignant cells. MRI combined with PET-FDG imaging provides improved detection of underlying pathologies. DCE-MRI involves serial acquisition of MR images in regions of interest before, during, and after intravenous injection of contrast agent. Fitting of the data to pharmacokinetic models enables extract ion of physiological data, such as tissue perfusion, microvascular vessel wall permeability, and tissue cellularity, the latter correlating with the rate of water diffusion. For the study, comparisons of Kₜᵣₐₙₛ values between treatment groups are used as a metric for vascular permeability/integrity and treatment response. T₁ values are used to render tumor volume with values above 300 ms as pathologic. Changes in enhancement patterns and increases in rates of water diffusion following treatment represent changes in tissue cellularity and tumor vasculature integrity and are considered indicators of treatment responses.

Statistics. The means for three or more groups will be compared by one-way ANOVA. Tukey-Kramer multiple comparisons test will be used to identify differences between individual groups.

### REFERENCES

Ahrens et al., BioTechniques, 50:229 (2011).

Alizadeh et al., Nanomedicine, 6:382 (2010).

Apetoh et al., Nat. Med., 13:1050 (2007).

Ashley et al., ACS Nano, 6:2174 (2012).

Ashley et al., Nat. Mater., 10:389 (2011).

Bahadar et al., Iran. Biomed. J 20:1 (2016).

Basu et al., Immunity, 14:303 (2001).

Bezu et al., Front. Immunol., 6:187 (2015).

Bhattacharyya et al., Nat. Commun. 6:7939 (2015).

Black et al., Oncotarget, 7:10557 (2016).

Brabazon et al., J. Neurotrauma. Ahead of Print.

Broz et al., Cancer Cell, 26:638 (2014).

Brust et al., Eur. J. Nucl. Med., 28:721 (2001).

Casares et al., J. Exp. Med., 202:1691 (2005).

Choi et al., Biomaterials, 33:4195 (2012).

Croissant et al., Adv. Mater., 26:6174 (2014).

Croissant et al., Adv. Mater, 27:145 (2015).

Croissant et al., Angew Chem. Int. Ed. Engl., 52:13813 (2013).

Croissant et al., J. Am. Chem. Soc., 134:7628 (2012).

Dasari & Tchounwou, Euro. J. Pharma., 0:364 (2014).

Drummond et al., Cancer Res., 66:3271 (2006).

Dunn et al., Annu. Rev. Immunol., 22:329 (2004).

Epler et al., Adv, healthcare Mater., 1:348 (2014

Espey, Free Radic. Biol. Med., 41:1621 (2006).

Faivre et al., Biochem. Pharma., 66:225 (2003).

Fluck et al., Microbiol. Mol. Biol. Rev., 73:542 (2009).

Francis et al., Adv. Drug Deliv. Rev., 114:33 (2017).

Frese et al., Cancer Discov., 2:260 (2012).

Fucikova et al., Cancer Res., 71:4821 (2011).

Gabizon et al., Olin. Pharmacokinet., 42:419 (2003).

Gabizon et al., J. Drug Target, 10:539 (2002).

Gardai et al., Cell, 115:13 (2003).

Gnjatic et al., Cancer J., 16:382 (2010).

Goel et al., Adv. Drug Deliv. Rev., 113:157 (2017).

Guiducci et al., Cancer Res., 65:3437 (2005).

Hackman et al., Mol. Imaging, 1:36 (2002),

Hamid et al New Ella, J. Med., 369:134 (2013),

Hato et al., Clin. Cancer Res., 20:2831 (2014).

He et al., Nat, Commun., 7:12499 (2016).

Higuchi et al., Cancer Immunol. Res., 3:1257 (2015),

Hingorani et al., Cancer Cell, 7:469 (2005).

Hitchens of al., Soc. May. Reso. Medicine, 65:1144 (2011).

Hou et al., Cancer Res., 67:792 (2007).

Hrkach et al., Sci. Transl. Med., 4:128ra139 (2012).

https://www.cancer.org/cancer/breast-cancer/about.html.

Ilyinskii et al., Vaccine, 32:2882 (2014).

Jia et al., Food Chem. Toxicol., 46:203 (2008).

Kalra et al., Cancer Res. 74: 2065 (2014),

Kauppinen, NMR Biomed., 15:6 (2002).

Kim et al., Proc. Nati Acad. Sci. USA, 113:4027 (2016).

Klimp et al., Crit. Rev. Oncol. Hematol., 44:143 (2002).

Koido et al., Clin. Cancer Res., 20:4228 (2014).

Kroemer et al., G., Annu. Rev. Immunol. 31,51-72 (2013).

Lancet et al., Blood, 123:3239 (2014).

Lee et al., Nat. Med., 20:152 (2014).

Lee et al., Proc. Natl. Acad. Sci, USA, 103:16649 (2006).

Li et al., Chem, Soc, Rev., 41:2590 (2012).

Li et al., J. Controlled Release Sac., 268:128 (2017).

Lin et al., Am. J. Pathol., 163:2113 (2003).

Litzenburger et al., Oncotarget, 5:1038 (2014).

Liu et al., ACS Nano, 10:2702 (2016).

Liu et al., J. Olin. Invest., 127:2007 (2017).

Lob et al., Nat. Rev. Cancer, 9:445 (2009). Ma et al., J. Exp. Med., 208:491 (2011).

Mall et al., Oncoimmunology, 5:e1075114 (2016).

Martins et al., Oncogene, 30:1147 (2011),

McCormick et al., Hum. Vaccines lmmunoiher., 12:563 (2016),

Meng et al., ACS Nano, 5:4131 (2011),

Meng et al., ACS Nano, 7:10048 (2013).

Meng et al., ACS Nano, 7:994 (2013).

Meng et al., ACS Nano, 9:3540 (2015).

Meraz et al., Molecular Pharm., 11:3484 (2014).

Meraz et al., Molecular Pharm., 9:2049 (2012).

Metz et al., Oncoimmunology, 1:1460 (2012),

Michaud el al., Science, 334:1573 (2011).

Napolitani et al., Nat. Immunol., 6:769 (2005).

Noureddine et al., Chemistry, 22:9624 (2016).

Noureddine et al., Nanoscale, 7:11444 (2015).

Obeid et al., Cell Death and Differentiation, 14:1848 (2007).

Obeid et al., Nat. Med., 13:54 (2007).

Pang et al., Oncotarget, 7:37081 (2016).

Paniccia et al., Chinese J. Cancer Res., 27:376 (2015).

Pardoll, Nat. Rev. Cancer, 12:252 (2012).

Passero et al., Expert Rev. Anticancer Ther., 16:697 (2016).

Pawaria et al., Nat. Commun., 2:521 (2011).

Pfirschke et al., Immunity, 44:343 (2016).

Pokharkar et al., J. Biomed. Nanotechnol., 5:233 (2009).

Prendergast et al., Cancer Immunol., Immunother., 63:721 (2014),

Provenzano et al., Br. J. Cancer, 108:1 (2013).

Radu et al., Nat, Med., 14:783 (2008).

Reddy et al., Biomed Pap, Med. Fac. Univ. Palacky Olomouc Czech Repub., 148:161 (2004).

Roers et al., Immunity, 44:739 (2016).

Ronen et al., Mol. Ther., 19:1273 (2011).

Rossi et al., J. Clin. Oncol., 32:3029 (2014).

Shao et al., J. Immunol., 171:4780 (2003).

Shi et al., Mol. Immunol., 66:208 (2015).

Siegel et al., Cancer J. Clin., 64:9 (2014).

Solheim et al., Cancer Gene Ther. 14:364 (2007).

Soliman et al., Oncotarget, 5:8136 (2014).

Soliman et al., Oncotarget, 7:22928 (2016).

Soma et al., J. Controlled Rel. Soc., 68:283 (2000).

Soundararajan et al., Nuclear Med. Biol., 36:515 (2009).

Sparreboom et al., Clin. Cancer Res., 11:4136 (2005).

Spranger et al., Nature, 523:231 (2015).

Springer el al., NMR in Biomed., 27:760 (2014).

Strug et al., J. Proteome Res., 7:2703 (2008).

Tavare et al., Cancer Res., 76:73 (2016).

Tavare et al., J. Nuclear Med., 56:1258 (2015).

Tesniere et al., Oncogene, 29:482 (2010).

Torres et al., PLoS ONE 8:e80580 (2013).

Tseng et al., Clin. Cancer Res., 16:3684 (2010).

Vacchelli et al., Oncoimmunology, 3:e957994 (2014).

Valable et al., Neuroimage, 40:973 (2008).

Verra et al., Eur. Urol., 48:527 (2005).

Wang et al., Cancer Immuno. Res., 2:846 (2014).

Wheeler et al., Int. Arch. Allergy Immunol., 126:135 (2001).

Xia et al., ACS Nano, 3:3273 (2009),

Yankeelov et al., Magnetic Reson. Imag., 25:1 (2007),

Yotsumoto et al., J. Immunol., 180:809 (2008).

Zappasodi et al., Cancer Res., 70:9062 (2010).

Zhang et al., ACS Applied Materials & Interfaces, 8:13332 2016.

Zhao et al., Biomaterials, 102:187 (2016).

Zhao et al., J. Intl. du Cancer, 129:2651 (2011).

Zhao et al., Mol. Cell. Biochem., 359:217 (2012).

Zhuang et al., Onco Targets Ther., 6:549 (2013).

Zou, Nat. Rev. Cancer, 5:263 (2005).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A mesoporous hybrid siliceous nanoparticle comprising an immune stimulant and an agent to induce immunogenic cell death (ICD).

2. The nanoparticle of claim 1 wherein the immune stimulant comprises a TLR4 ligand.

3. (canceled)

4. The nanoparticle of claim 2 further comprising a TLR9 ligand.

5. The nanoparticle of claim 4 wherein the TLR9 ligand comprises CpG oligonucleotides, SD-101AS15, GNKG168, PF-3512676, ISS 1018, IMO-2055, CpG-28, EMD120108, or BCG.

6-8. (canceled)

9. The nanoparticle of claim 2 wherein the TLR4 ligand comprises monophosphoryl lipid (MPL)-A, aminoalkyl glucosaminide phosphate (AGP), glucopyranosyl LPS, beta-defensin 2, fibronectin EDA, HMGB1, AS15, snapin, tenascin C, ER111232, ER111233, ER112040, ER111230, ER112231, ER112093, ER112049, ER112047, ER112066, ER113651, ER119327, ER803022, ER803732, or ER803789.

10. The nanoparticle of claim 1 wherein the agent that induces ICD comprises an anthracycline, R2016 (3-(4-chlorophenylamino)-6-hydroxy-9-methyl-9H-carbazole-1,4-dione), an anthracenedione, a platinin, an alkylating agent, proteasomal inhibitor, or immunogenic cell-killing RNA.

11-12. (canceled)

13. The nanoparticle of claim 1 wherein the agent that induces ICD is linked to the silaceous core.

14. The nanoparticle of claim 13 wherein the linker is pH sensitive, light sensitive, redox sensitive or comprises a hydrazine or benzene-bridged silsesquioxane.

15. The nanoparticle of claim 1 which has a diameter of about 50 nm to about 150 nm or about 75 um to about 300 nm or which has pores of about 5 to 20 nm in diameter or about 8 to 15 nm in diameter.

16-18. (canceled)

19. The nanoparticle of claim 1 further comprising a lipid layer.

20-27. (canceled)

28. A method to stimulate antitumor immunity, activate dendritic cells (DCs), or stimulate antigen processing presentation in a mammal, comprising administering to the mammal an effective amount of a composition comprising a plurality of the nanoparticles of claim 1, and a checkpoint inhibitor.

29. The method of claim 28 wherein the checkpoint inhibitor is an anti-PD1 agent.

30. The method of claim 28 wherein the mammal is a human.

31. The method of claim 28, wherein the mammal has cancer.

32. The method of claim 31 wherein the cancer is kidney, liver, lung, ovary, pancreas, breast, brain, bladder, stomach, or prostate cancer, leukermia or lymphoma.

33. (canceled)

34. The method of claim 29 wherein the anti-PD1 agent comprises dinaciclib, nivolumab, pembrolizumab, pidilizumab, BMS-936559, MPDL3280A, MEDI4736, MSB0010718C, avelumab, durvalumab, or atezolizumab

35. The method of claim 28 further comprising administering IL-12.

36. The method of claim 28 wherein the composition is injected.

37. The method of claim 28 wherein the composition is subcutaneously or intratumorally administered.

38. The method of claim 28 wherein the composition is systemically administered.

39-40. (canceled)


SEQUENCE LISTING (11 sequences)

SEQ ID NO: 1 - protein, 8 aa, Artificial Sequence
  A synthetic amino acid sequence
RRRRRRRR

SEQ ID NO: 2 - protein, 25 aa, Artificial Sequence
  A synthetic amino acid sequence
GLFHAIAHFIHGGWHGLIHGWYGGC

SEQ ID NO: 3 - protein, 8 aa, Artificial Sequence
  A synthetic amino acid sequence
RRRRRRRR

SEQ ID NO: 4 - protein, 30 aa, Artificial Sequence
  A synthetic amino acid sequence
WEARLARALARALARHLARALARALRAGEA

SEQ ID NO: 5 - protein, 30 aa, Artificial Sequence
  A synthetic amino acid sequence
WEAKLAKALAKALAKHLAKALAKALKAGEA

SEQ ID NO: 6 - protein, 30 aa, Artificial Sequence
  A synthetic amino acid sequence
WEAALAEALAEALAEHLAEALAEALEALAA

SEQ ID NO: 7 - protein, 23 aa, Artificial Sequence
  A synthetic amino acid sequence
GLFEAIEGFIENGWEGMIDGWYG

SEQ ID NO: 8 - protein, 30 aa, Artificial Sequence; identical to SEQ ID NO: 4
  A synthetic amino acid sequence

SEQ ID NO: 9 - protein, 30 aa, Artificial Sequence; identical to SEQ ID NO: 5
  A synthetic amino acid sequence

SEQ ID NO: 10 - protein, 30 aa, Artificial Sequence; identical to SEQ ID NO: 6
  A synthetic amino acid sequence

SEQ ID NO: 11 - protein, 23 aa, Artificial Sequence
  A synthetic amino acid sequence
GLFEAIEGFIENGWEGMIDGWYG